(19) United States
(12) Patent Application Publication
(10) Pub. No.: US 2025/0277231 A1
(43) Pub. Date: Sep. 4, 2025

(54) METHOD FOR GENERATING T-CELLS COMPATIBLE FOR ALLOGENIC TRANSPLANTATION

(71) Applicant: CELLECTIS, Paris (FR)

(72) Inventors: Laurent POIROT, Paris (FR); David SOURDIVE, Levallois-Perret (FR); Philippe DUCHATEAU, Dravell (FR); Jean-Pierre CABANIOLS, Saint Lau la Foret (FR)

(73) Assignee: CELLECTIS, Paris (FR)

(21) Appl. No.: 19/067,358

(22) Filed: Feb. 28, 2025

Related U.S. Application Data
(62) Division of application No. 18/480,890, filed on Oct. 4, 2023, now Pat. No. 12,252,699.

(30) Foreign Application Priority Data
Mar. 11, 2014 (DK) PA201470119

(51) Int. Cl. C12N 15/85; A61K 35/17; A61K 39/00; C07K 14/705; C07K 14/74; C12N 5/0783; C12N 15/113; C12N 15/90

(52) U.S. Cl. CPC C12N 15/85; A61K 35/17; A61K 39/0011; C07K 14/70503; C07K 14/70539; C12N 5/0636; C12N 15/1138; C12N 15/907; A61K 2039/5156; A61K 2039/5158; C07K 2317/24; C07K 2317/622

(57) ABSTRACT

The present invention pertains to engineered T-cells, method for their preparation and their use as medicament, particularly for immunotherapy. The engineered T-cells of the invention are characterized in that the expression of beta 2-microglobulin (B2M) and/or class II major histocompatibility complex transactivator (CIITA) is inhibited, e.g., by using rare-cutting endonucleases able to selectively inactivating by DNA cleavage the gene encoding B2M and/or CIITA, or by using nucleic acid molecules which inhibit the expression of B2M and/or CIITA. In order to further render the T-cell non-alloreactive, at least one gene encoding a component of the T-cell receptor is inactivated, e.g., by using a rare-cutting endonucleases able to selectively inactivating by DNA cleavage the gene encoding said TCR component. In addition, expression of immunosuppressive polypeptide can be performed on those modified T-cells in order to prolong the survival of these modified T cells in host organism. Such modified T-cell is particularly suitable for allogeneic transplantations, especially because it reduces both the risk of rejection by the host's immune system and the risk of developing graft versus host disease. The invention opens the way to standard and affordable adoptive immunotherapy strategies using T-Cells for treating cancer, infections and auto-immune diseases.

### CROSS REFERENCE TO RELATED APPLICATIONS

This application is a divisional of U.S. application Ser. No. 18/480,890 filed on Oct. 4, 2023, which is a divisional of U.S. application Ser. No. 16/138,908 filed on Sep. 21, 2018, now U.S. Pat. No. 11,820,996, which is a divisional of U.S. application Ser. No. 15/123,974 filed on Sep. 6, 2016 now abandoned, which is a U.S. National Stage of PCT/EP2015/055097 filed on Mar. 11, 2015, which claims the benefit of Danish Application PA201470119 filed on Mar. 11, 2014, all of which are herein incorporated by reference in their entirety.

The instant application contains a Sequence Listing which has been submitted electronically in XML format and is hereby incorporated by reference in its entirety. Said XML copy, created on Sep. 28, 2023, is named DI2013-25US3_SL.xml and is 155,648 bytes in size.

### FIELD OF THE INVENTION

The present invention pertains to engineered T-cells, method for their preparation and their use as medicament, particularly for immunotherapy. The engineered T-cells of the invention are characterized in that the expression of beta 2-microglobulin (B2M) and/or class II major histocompatibility complex transactivator (CIITA) is inhibited, e.g., by using rare-cutting endonucleases able to selectively inactivating by DNA cleavage the gene encoding B2M and/or CIITA, or by using nucleic acid molecules which inhibit the expression of B2M and/or CIITA. In order to further render the T-cell non-alloreactive, at least one gene encoding a component of the T-cell receptor is inactivated, e.g., by using a rare-cutting endonucleases able to selectively inactivating by DNA cleavage the gene encoding said TCR component. In addition, a step of expression of immunosuppressive polypeptide such as viral MHCI homolog or NKG2D ligand can be performed on those modified T-cells in order to prolong the survival of these modified T cells in host organism. Such modified T-cell is particularly suitable for allogeneic transplantations, especially because it reduces both the risk of rejection by the host's immune system and the risk of developing graft versus host disease. The invention opens the way to standard and affordable adoptive immunotherapy strategies using T-Cells for treating cancer, infections and auto-immune diseases.

### BACKGROUND OF THE INVENTION

Adoptive immunotherapy, which involves the transfer of autologous antigen-specific T-cells generated ex vivo, is a promising strategy to treat viral infections and cancer. The T-cells used for adoptive immunotherapy can be generated either by expansion of antigen-specific T-cells or redirection of T-cells through genetic engineering (Park, Rosenberg et al. 2011). Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T-cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors.

The current protocol for treatment of patients using adoptive immunotherapy is based on autologous cell transfer. In this approach, T lymphocytes are recovered from patients, genetically modified or selected ex vivo, cultivated in vitro in order to amplify the number of cells if necessary and finally infused into the patient. In addition to lymphocyte infusion, the host may be manipulated in other ways that support the engraftment of the T cells or their participation in an immune response, for example pre-conditioning (with radiation or chemotherapy) and administration of lymphocyte growth factors (such as IL-2). Each patient receives an individually fabricated treatment, using the patient's own lymphocytes (i.e. an autologous therapy). Autologous therapies face substantial technical and logistic hurdles to practical application, their generation requires expensive dedicated facilities and expert personnel, they must be generated in a short time following a patient's diagnosis, and in many cases, pretreatment of the patient has resulted in degraded immune function, such that the patient's lymphocytes may be poorly functional and present in very low numbers. Because of these hurdles, each patient's autologous cell preparation is effectively a new product, resulting in substantial variations in efficacy and safety.

Ideally, one would like to use a standardized therapy in which allogeneic therapeutic cells could be pre-manufactured, characterized in detail, and available for immediate administration to patients. By allogeneic it is meant that the cells are obtained from individuals belonging to the same species but are genetically dissimilar. However, the use of allogeneic cells presently has many drawbacks. In immune-competent hosts allogeneic cells are rapidly rejected, a process termed host versus graft rejection (HvG), and this substantially limits the efficacy of the transferred cells. In immune-incompetent hosts, allogeneic cells are able to engraft, but their endogenous T-cell receptors (TCR) specificities may recognize the host tissue as foreign, resulting in graft versus host disease (GvHD), which can lead to serious tissue damage and death.

In order to provide allogeneic T-cells, the inventors previously disclosed a method to genetically engineer T-Cells, in which different effector genes, in particular those encoding T-cell receptors, were inactivated by using specific TAL-nucleases, better known under the trade mark TALENT (Cellectis, 8, rue de la Croix Jarry, 75013 PARIS). This method has proven to be highly efficient in primary cells using RNA transfection as part of a platform allowing the mass production of allogeneic T-cells (WO 2013/176915).

Beta-2 microglobulin, also known as B2M, is the light chain of MHC class I molecules, and as such an integral part of the major histocompatibility complex In human, B2M is encoded by the b2m gene which is located on chromosome 15, opposed to the other MHC genes which are located as gene cluster on chromosome 6. The human protein is composed of 119 amino acids (SEQ ID NO: 1) and has a molecular weight of 11.800 Daltons. Mice models deficient for beta-2 microglobulin have shown that B2M is necessary for cell surface expression of MHC class I and stability of the peptide binding groove. It was further shown that haemopoietic transplants from mice that are deficient for normal cell-surface MHC I expression are rejected by NK1.1+ cells in normal mice because of a targeted mutation in the beta-2 micorglobulin gene, suggesting that deficient expression of MHC I molecules renders marrow cells susceptible to rejection by the host immune system (Bix et al. 1991).

CIITA protein (SEQ ID NO: 4-NCBI Reference Sequence: NP_000237.2) that acts as a positive regulator of class II major histocompatibility complex gene transcription, including β2m gene transcription, and is often referred to as the “master control factor” for the expression of these genes. CIITA mRNA (SEQ ID NO: 5) can only be detected in human leukocyte antigen (HLA) system class II-positive cell lines and tissues. This highly restricted tissue distribution suggests that expression of HLA class II genes is to a large extent under the control of CIITA (Mach B., et al. 1994).

Adaptive immune response is a complex biological system where numerous cellular components interact. Professional Antigen Presenting Cells (APC) are able to process foreign bodies and expose them to helper T cells in the context of MHC Class II molecules. Activated helper T cells will in turn stimulate B cells response and cytotoxic T (CTL) cells response. CTL recognize foreign peptides presented by MHC Class I molecules but in the case of alloreactivity, recognize and kill cells bearing foreign MHC Class I. MHC Class I molecules are composed of 2 entities: the highly polymorphic, transmembrane heavy chain and a small invariant polypeptide, the beta2-microglobuline (beta2-m) encoded by B2M gene. The expression of the MHC Class I heavy chain at the cell surface requires its association with the beta2-m. Hence, abrogation of beta2-m expression in CAR T cells will impair MHC Class I expression and make them invisible to host CTL. However, MHC Class I deficient CAR T cells are susceptibe to lysis by host NK cells, which target cells lacking MHC Class I molecules [Ljunggren HG et al. (1990), Immunl Today. 11:237-244].

NK cells exert cytotoxic functions towards the cells they interact with based on the balance between activating and inhibitory signals they received through different monomorphic or polymorphic receptors. One central activating receptor on human NK cells is NKG2D and its ligands include proteins such as MICA, MICB, ULBP1, ULBP2, ULBP3 [Raulet DH, (2003), Nature Reviews Immunology 3 (10): 781-79]. On the other hand, the inhibitory signal is mediated through the interaction between NK receptors like LIR-1/ILT2 and MHC Class I molecules [Ljunggren HG et al. (1990), Immunl Today. 11:237-244]. Some viruses such as cytomegaloviruses have aquired mechanisms to avoid NK cell mediate immune surveillance. HCMV genome encodes proteins that are able to prevent MHC Classl surface expression (i.e. US2, US3, US6 and US11) while expressing a MHC classl homolog protein (UL18) that acts as a decoy to block NK-mediated cell lysis [Kim, Y et al. (2008), PLOS Pathogens. 4: e1000123, and Wilkinson G. et al. (2010). J Clin Virol. 41 (3): 206-212]. Moreover, HCMV interferes with the NKG2D pathway by secreting a protein able to bind NKG2D ligands and prevent their surface expression [Welte SA et al. (2003), Eur J Immunol 33 (1): 194-203]. In tumor cells, some mechanisms have evolved to evade NKG2D response by secreting NKG2D ligands such as ULBP2, MICB or MICA (Waldhauer I, Steinle A (2003). Proteolytic release of soluble UL16-binding protein 2 from tumor cells. Cancer Res 2006; 66 (5): 2520-2526; Salih HR et al. (2006), Hum Immunol. 2006 March; 67 (3): 188-95; Salih HR et al. (2003) Blood. 2003 Aug. 15; 102 (4): 1389-96; Salih HR et al. (2002) J Immunol.; 169 (8): 4098-102].

The present inventor here provides strategies for immunotherapy by which T-cells, especially allogeneic T-cells, are made particular suitable for allogeneic transplantations, reducing the risk for host versus graft rejections and for developing graft versus host disease and to render the T cells “stealthy”, in particular with respect to APC cells or NK cells.

### SUMMARY OF THE INVENTION

The present invention concerns methods for preparing engineered T-cells, in particular allogeneic T-cells obtained from a donor, to make them suitable for immunotherapy purposes. The methods of the present invention more particularly allow the precise modulation of expression of certain effector molecules important for immune recognition and histocompatibility.

According to one aspect, the present invention provides a method for preparing an engineered T-cell, preferably an allogeneic T-cell obtained from a donor, comprising the steps of:
a) providing a T-cell, preferably an allogeneic T-cell obtained from a donor; and
b) inhibiting the expression of beta 2-microglobulin (B2M) and/or class II major histocompatibility complex transactivator (CIITA) in said T-cell.

According to certain embodiments, inhibition of expression of B2M is achieved by a genome modification, more particularly through the expression in the T-cell of a rare-cutting endonuclease able to selectively inactivate by DNA cleavage the gene encoding B2M, such as the human β2m gene set forth in SEQ ID NO: 2 (NCBI Reference Sequence: NG_012920.1), or a gene having at least 70%, such as at least 80%, at least 90% at least 95%, or at least 99%, sequence identify with the human β2m gene set forth in SEQ ID NO: 2 over the entire length of SEQ ID NO: 2. Such rare-cutting endonuclease may be a TAL-nuclease, meganuclease, zing-finger nuclease (ZFN), or RNA guided endonuclease (such as Cas9).

According to certain other embodiments, inhibition of expression of B2M is achieved by using (e.g., introducing into the T-cell) a nucleic acid molecule that specifically hybridizes (e.g. binds) under cellular conditions with the cellular mRNA and/or genomic DNA encoding B2M, thereby inhibiting transcription and/or translation of the gene. In accordance with particular embodiments, the inhibition of expression of B2M is achieved by using ((e.g., introducing into the T-cell) an antisense oligonucleotide, ribozyme or interfering RNA (RNAi) molecule. Preferably, such nucleic acid molecule comprises at least 10 consecutive nucleotides of the complement of SEQ ID NO: 3 (i.e., the mRNA encoding human B2M; NCBI Reference Sequence: NM_004048).

According to certain embodiments, inhibition of expression of CIITA is achieved by a genome modification, more particularly through the expression in the T-cell of a rare-cutting endonuclease able to selectively inactivate by DNA cleavage the gene encoding CIITA, such as the human CIITA gene (NCBI Reference Sequence: NG_009628.1), or a gene having at least 70%, such as at least 80%, at least 90% at least 95%, or at least 99%, sequence identify with the human CIITA gene according to NG_009628.1 over the entire length of the human CIITA gene according to NG_009628.1. Such rare-cutting endonuclease may be a TAL-nuclease, meganuclease, zing-finger nuclease (ZFN), or RNA guided endonuclease (such as Cas9).

According to certain other embodiments, inhibition of expression of CIITA is achieved by using (e.g., introducing into the T-cell) a nucleic acid molecule that specifically hybridizes (e.g. binds) under cellular conditions with the cellular mRNA and/or genomic DNA encoding CIITA, thereby inhibiting transcription and/or translation of the gene. In accordance with particular embodiments, the inhibition of expression of CIITA is achieved by using ((e.g., introducing into the T-cell) an antisense oligonucleotide, ribozyme or interfering RNA (RNAi) molecule. Preferably, such nucleic acid molecule comprises at least 10 consecutive nucleotides of the complement of SEQ ID NO: 5 (i.e., the mRNA encoding human CIITA isoform 2).

According to particular embodiments, the T-cell may be further engineered to make it non-alloreactive, especially by inactivating one or more genes involved in self-recognition, such as those, for instance, encoding components of T-cell receptors (TCR). This can be achieved by a genome modification, more particularly through the expression in the T-cell of a rare-cutting endonuclease able to selectively inactivate by DNA cleavage, preferably double-strand break, at least one gene encoding a component of the T-Cell receptor (TCR), such as the gene encoding TCR alpha or TCR beta. Such rare-cutting endonuclease may be a TAL-nuclease, meganuclease, zing-finger nuclease (ZFN), or RNA guided endonuclease (such as, Cas9). Preferably, the rare-cutting endonuclease is able to selectively inactivate by DNA cleavage the gene coding for TCR alpha.

According to optional embodiments, the T-cell may be further engineered to express a Chimeric Antigen Receptor (CAR) directed against at least one antigen expressed at the surface of a malignant or infected cell, such as the B-lymphocyte antigen CD19.

The present invention thus provides in a further aspect engineered T-cells, in particular engineered isolated T-cells, characterized in that the expression of beta 2-microglobulin (B2M) is inhibited.

According to certain embodiments, a T-cell is provided which expresses a rare-cutting endonuclease able to selectively inactivate by DNA cleavage the gene encoding B2M. More particularly, such T-cell comprises an exogenous nucleic acid molecule comprising a nucleotide sequence encoding said rare-cutting endonuclease, which may be a TAL-nuclease, meganuclease, zing-finger nuclease (ZFN), or RNA guided endonuclease.

According to certain other embodiments, a T-cell is provided which comprises an exogenous nucleic acid molecule that inhibits the expression of B2M. According to particular embodiments, such nucleic acid molecule is an antisense oligonucleotide, ribozyme or interfering RNA (RNAi) molecule. According to preferred embodiments, such nucleic acid molecule comprises at least 10 consecutive nucleotides of the complement of SEQ ID NO: 3.

The present invention further provides engineered T-cells, in particular engineered isolated T-cells, characterized in that the expression of class II major histocompatibility complex transactivator (CIITA) is inhibited.

According to certain embodiments, a T-cell is provided which expresses a rare-cutting endonuclease able to selectively inactivate by DNA cleavage the gene encoding CIITA. More particularly, such T-cell comprises an exogenous nucleic acid molecule comprising a nucleotide sequence encoding said rare-cutting endonuclease, which may be a TAL-nuclease, meganuclease, zing-finger nuclease (ZFN), or RNA guided endonuclease.

According to certain other embodiments, a T-cell is provided which comprises an exogenous nucleic acid molecule that inhibits the expression of CIITA. According to particular embodiments, such nucleic acid molecule is an antisense oligonucleotide, ribozyme or interfering RNA (RNAi) molecule. According to preferred embodiments, such nucleic acid molecule comprises at least 10 consecutive nucleotides of the complement of SEQ ID NO: 5.

According to particular embodiments, the T-cell may further have at least one inactivated gene encoding a component of the TCR receptor. More particularly, such T-cell may express a rare-cutting endonuclease able to selectively inactivate by DNA cleavage, preferably double-strand break, said at least one gene encoding a component of the T-Cell receptor (TCR). Accordingly, said T-cell may comprise an exogenous nucleic acid molecule comprising a nucleotide sequence coding for a rare-cutting endonuclease able to selectively inactivate by DNA cleavage at least one gene coding for one component of the T-Cell receptor (TCR). The disruption of TCR provides a non-alloreactive T-cell that can be used in allogeneic treatment strategies.

According to optional embodiments, the T-cell may be engineered to express a Chimeric Antigen Receptor (CAR) directed against at least one antigen expressed at the surface of a malignant or infected cell, such as the B-lymphocyte antigen CD19. Particularly, the T-cell comprises an exogenous nucleic acid molecule comprising a nucleotide sequence encoding said CAR. The binding of the target antigen by the CAR has the effect of triggering an immune response by the T-cell directed against the pathological cell, which results in degranulation of various cytokine and degradation enzymes in the interspace between the cells.

According to some embodiments, an additional modification of T-cells is performed to render them stealthy by expression of at least one non-endogenous immunosuppressive polypeptide such as a viral MHC homolog, for instance, UL18, or such as a NKG2D ligand.

According to some embodiments, the T-cell of the present invention expresses at least one non-endogenous immune-suppressive polypeptide. According to more particular embodiments, said non-endogenous immune-suppressive polypeptide is a viral MHC homolog, such as UL18. The T-cell may comprise an exogenous nucleic acid molecule comprising a nucleotide sequence cording for a polypeptide sharing at least 80%, preferably at least 90% and more preferably at least 95% of identity with SEQ ID NO: 89. According to other more particular embodiments, said non-endogenous immune-suppressive polypeptide is a NKG2D ligand. The T-cell may comprise an exogenous nucleic acid molecule comprising a nucleotide sequence cording for a polypeptide sharing at least 80%, preferably at least 90% and more preferably at least 95% of identity with any one of SEQ ID NO: 90-97.

As a result of the present invention, engineered T-cells can be used as therapeutic products, ideally as an “off the shelf” product, for use in the treatment or prevention cancer, bacterial or viral infections, or auto-immune diseases.

Thus, the present invention further provides an engineered T-cell or a composition, such as a pharmaceutical composition, comprising same for use as a medicament. According to certain embodiments, the engineered T-cell or composition is for use in the treatment of a cancer, and more particularly for use in the treatment of lymphoma. According to certain other embodiments, the engineered T-cell or composition is for use in the treatment of viral infection. According to certain other embodiments, the engineered T-cell or composition is for use in the treatment of bacterial infection.

It is understood that the details given herein with respect to one aspect of the invention also apply to any of the other aspects of the invention.

### BRIEF DESCRIPTION OF THE DRAWINGS

FIG. 1: Schematic representation of the normal relationship between donor's T-cells, host T-cells and antigen presenting cells.

FIG. 2: Schematic representation of the genetically modified therapeutic T-cells according to the invention and the patient's T-cells and tumor cells.

FIG. 3: Comparison of the forward side scatter (FSC) distribution, an indicator of cell size, between TCR-positive and TCR-negative cells.

FIG. 4: Flow cytometry analysis of TCR alpha/beta and CD3 expression on human primary T cells following TRAC TALE-nuclease mRNA electroporation (top).

FIG. 5A-B: Flow cytometry analysis of HLA_ABC expression on the surface of human primary T cells in: A. Control T-cells. B. following B2m TALE-nuclease mRNA electroporation.

FIG. 6A-B: A. Flow cytometry analysis of CAR expression (anti F(ab′)2) after electroporation of T cells with or without mRNA encoding a single chain CAR. B. Flow cytometry analysis of CD107a expression (marker of degranulation) on electroporated T cells cocultured with daudi cells.

FIG. 7: Schematic representation of the potential interactions between an allogeneic CAR T cell with diverse host immune cells (CD8+ and CD4+ T cell, APC such as dendritic cell and NK cell), the CAR T cell having its B2M gene inactivated by KO. Sign (+) represents activation and sign (−) inhibition. The potential interaction between CAR T cell with the tumor cell remains unchanged. The inactivation of B2M gene which is one component of the MCHI, renders the latter non-functional in regards to the interactions with host cytotoxic T cell (CD8+) and with NK cell. Then, NK cell can exert its activation on allogeneic CAR T cell via activator pathway such NKG2D/NKG2D ligand.

FIG. 8: Schematic representation of the potential interactions between an allogeneic CAR T cell with diverse host immune cells (CD8+ and CD4+ T cell, APC such as dendritic cell and NK cell), the CAR T cell having its B2M gene inactivated by KO and expressing viral MHCI homolog. Sign (+) represents activation and sign (−) inhibition. The potential interaction between CAR T cell with the tumor cell remains unchanged. As for the preceding figure (only B2M KO), the interaction between CAR T cell and host CD8+ T cell is alleviated. In this case, the expression of viral MHCI homolog renders the interaction with NK cell inoperative via MHCI/inhibitor receptor. The double genetic modification of allogeneic CAR T cells by KO of B2M combined with the expression of viral MHCI homolog strengthens their immunosuppressive protection.

FIG. 9: Schematic representation of the potential interactions between an allogeneic CAR T cell with diverse host immune cells (CD8+ and CD4+ T cell, APC such as dendritic cell and NK cell), the CAR T cell having its B2M gene inactivated by KO and expressing a soluble NKG2D ligand. Sign (+) represents activation and sign (−) inhibition. The potential interaction between CAR T cell with the tumor cell remains unchanged. As for the preceding figure (only B2M KO), the interaction between CAR T cell and host CD8+ T cell is alleviated. The expression of soluble NKG2D ligand is another way to inactivation the interaction with NK cell. In this case, the soluble NKG2D ligand can bind to NKG2D receptor on NK cell but exerts no action, in contrast to the NKG2D ligand of CAR T cell with which it exerts an inhibitory competition. The double genetic modification of allogeneic CAR T cells by KO of B2M combined with the expression of soluble NKG2D ligand strengthens their immunosuppressive protection.

FIG. 10: FACS analysis of B2-m expression in T cells. Untransfected (top) and transfected T cells (middle and bottom) are analysed by FACS for viability (left) and B2-m expression (right).

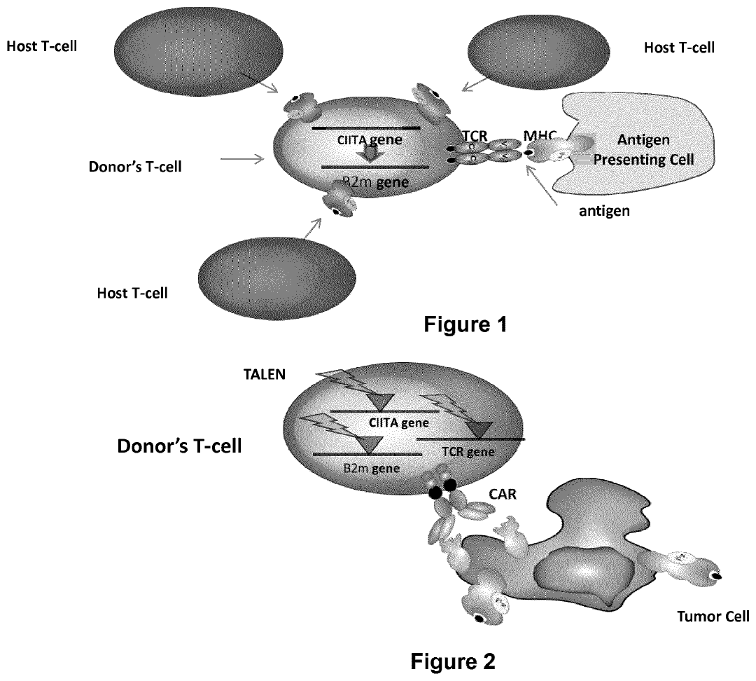

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Methods for Preparing Engineered T-Cells

In a general aspect, the present invention pertains to methods for preparing engineered T-cells, in particular allogeneic T-cells obtained from a donor.

Accordingly, the present invention provides a method for preparing an engineered T-cell, preferably an allogeneic T-cell obtained from a donor, said method comprises the steps of:
a) providing a T-cell, preferably an allogeneic T-cell obtained from a donor; and
b) inhibiting the expression of beta 2-microglobulin (B2M) and/or class II major histocompatibility complex transactivator (CIITA) in said T-cell.

According to certain embodiments, the method comprises inhibiting the expression of beta 2-microglobulin (B2M). Alternatively, or in addition, the method may comprise inhibiting the expression of class II major histocompatibility complex transactivator (CIITA).

According to certain embodiments, inhibition of expression of B2M is achieved by a genome modification, more particularly through the expression in the T-cell of a rare-cutting endonuclease able to selectively inactivate by DNA cleavage the gene encoding B2M (e.g. the human B2m gene set forth in SEQ ID NO: 2).

According to certain other embodiments, inhibition of expression of CIITA is achieved by a genome modification, more particularly through the expression in the T-cell of a rare-cutting endonuclease able to selectively inactivate by DNA cleavage the gene encoding CIITA (e.g. the human CIITA gene).

By “inactivating” or “inactivation of” a gene it is intended that the gene of interest (e.g. the gene encoding B2M or CIITA) is not expressed in a functional protein form. In particular embodiments, the genetic modification of the method relies on the expression, in provided cells to engineer, of a rare-cutting endonuclease such that same catalyzes cleavage in one targeted gene thereby inactivating said targeted gene. The nucleic acid strand breaks caused by the endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation or via the so-called microhomology-mediated end joining. Repair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art.

A rare-cutting endonuclease to be used in accordance of the present invention to inactivate the β2m gene may, for instance, be a TAL-nuclease, meganuclease, zing-finger nuclease (ZFN), or RNA guided endonuclease (such as Cas9).

According to a particular embodiment, the rare-cutting endonuclease is a TAL-nuclease.

According to another particular embodiment, the rate-cutting endonuclease is a homing endonuclease, also known under the name of meganuclease.

According to another particular embodiment, the rare-cutting endonuclease is a zing-finger nuclease (ZNF).

According to another particular embodiment, the rare-cutting endonuclease is a RNA guided endonuclease. According to a preferred embodiment, the RNA guided endonuclease is the Cas9/CRISPR complex.

According to a specific embodiment, the rare-cutting endonuclease is a TAL-nuclease encoded by a nucleic acid molecule comprising the nucleotide sequence set for in SEQ ID NO: 67. According to another specific embodiment, the rare-cutting endonuclease is a TAL-nuclease encoded by a nucleic acid molecule comprising the nucleotide sequence set for in SEQ ID NO: 68. In yet another specific embodiment, the rare-cutting endonuclease is a combination of a TAL-nuclease encoded by a nucleic acid molecule comprising the nucleotide sequence set for in SEQ ID NO: 67 and a TAL-nuclease encoded by a nucleic acid molecule comprising the nucleotide sequence set for in SEQ ID NO: 68.

In order to be expressed in the T-cell, said rare-cutting endonuclease may be introduced into the cell by way of an exogenous nucleic acid molecule comprising a nucleotide sequence encoding said rare-cutting endonuclease. According to particular embodiments, the method of the invention further comprises introducing into said T-cell an exogenous nucleic acid molecule comprising a nucleotide sequence coding for a rare-cutting endonuclease, preferably a rare-cutting endonuclease able to selectively inactivate by DNA cleavage the gene encoding B2M (e.g. the human β2m gene set forth in SEQ ID NO: 2). For example, the exogenous nucleic acid molecule may comprising the nucleotide sequence set for in SEQ ID NO: 67 or SEQ ID NO: 68.

As a result, an engineered T-cell is obtained which expresses a rare-cutting endonuclease, preferably a rare-cutting endonuclease able to selectively inactivate by DNA cleavage the gene encoding B2M. In consequence, inactivation of the B2M gene by said rare-cutting endonuclease leads to the inhibition of the expression of B2M in the engineered T-cell. Hence, an engineered T-cell is obtained which is characterized in that the expression of B2M is inhibited.

A rare-cutting endonuclease to be used in accordance of the present invention to inactivate the CIITA gene may, for instance, be a TAL-nuclease, meganuclease, zing-finger nuclease (ZFN), or RNA guided endonuclease (such as Cas9).

According to a particular embodiment, the rare-cutting endonuclease is a TAL-nuclease.

According to another particular embodiment, the rate-cutting endonuclease is a homing endonuclease, also known under the name of meganuclease.

According to another particular embodiment, the rare-cutting endonuclease is a zing-finger nuclease (ZNF).

According to another particular embodiment, the rare-cutting endonuclease is a RNA guided endonuclease. According to a preferred embodiment, the RNA guided endonuclease is the Cas9/CRISPR complex.

In order to be expressed in the T-cell, said rare-cutting endonuclease may be introduced into the cell by way of an exogenous nucleic acid molecule comprising a nucleotide sequence encoding said rare-cutting endonuclease. According to particular embodiments, the method of the invention further comprises introducing into said T-cell an exogenous nucleic acid molecule comprising a nucleotide sequence coding for a rare-cutting endonuclease, preferably a rare-cutting endonuclease able to selectively inactivate by DNA cleavage the gene encoding CIITA (e.g. the human CIITA gene).

As a result, an engineered T-cell is obtained which expresses a rare-cutting endonuclease, preferably a rare-cutting endonuclease able to selectively inactivate by DNA cleavage the gene encoding CIITA. In consequence, inactivation of the CIITA gene by said rare-cutting endonuclease leads to the inhibition of the expression of CIITA in the engineered T-cell. Hence, an engineered T-cell is obtained which is characterized in that the expression of CIITA is inhibited. According to certain other embodiments, inhibition of expression of B2M is achieved by using (e.g., introducing into the T-cell) a nucleic acid molecule that specifically hybridizes (e.g. binds) under cellular conditions with the cellular mRNA and/or genomic DNA encoding B2M, thereby inhibiting transcription and/or translation of the gene. In accordance with particular embodiments, the inhibition of expression of B2M is achieved by using (e.g., introducing into the T-cell) an antisense oligonucleotide, ribozyme or interfering RNA (RNAi) molecule.

According to a particular embodiment, the nucleic acid molecule is an antisense oligonucleotide.

According to other particular embodiments, the nucleic acid molecule is a ribozyme, preferably a hammerhead ribozyme.

According to other particular embodiments, the nucleic acid is an interfering RNA (RNAi) molecule, such as a micro RNA (miRNA), small interfering RNA (siRNA) or short hairpin RNA (shRNA). Hence, in accordance with a preferred embodiment, the nucleic acid molecule is a micro RNA. In accordance with another preferred embodiment, the nucleic acid molecule is a small interfering RNA. In accordance with another preferred embodiment, the nucleic acid molecule is a short hairpin RNA.

As a result, an engineered T-cell is obtained which is characterized in that the expression of B2M is inhibited.

Because B2M is an important structural component of the major histocompatibility complex (MHC), inhibition of B2M expression leads to a reduction or elimination of MHC molecules on the surface of the engineered T-cell. In consequence, the engineered T-cell no longer presents antigens on the surface which are recognized by CD8+ cells. Especially in case of an allogeneic T-cell obtained from a donor, reduction or elimination of nonself-antigen presenting MHC molecules on the surface of the T-cell prevents the engineered T-cell, when infused into an allogeneic host, from being recognized by the host CD8+ cells. This makes the engineered T-cell particular suitable for allogeneic transplantations, especially because it reduces the risk of rejection by the host's immune system.

According to certain other embodiments, inhibition of expression of CIITA is achieved by using (e.g., introducing into the T-cell) a nucleic acid molecule that specifically hybridizes (e.g. binds) under cellular conditions with the cellular mRNA and/or genomic DNA encoding CIITA, thereby inhibiting transcription and/or translation of the gene. In accordance with particular embodiments, the inhibition of expression of CIITA is achieved by using (e.g., introducing into the T-cell) an antisense oligonucleotide, ribozyme or interfering RNA (RNAi) molecule.

According to a particular embodiment, the nucleic acid molecule is an antisense oligonucleotide.

According to other particular embodiment, the nucleic acid molecule is a ribozyme, preferably a hammerhead ribozyme.

According to other particular embodiment, the nucleic acid is an interfering RNA (RNAi) molecule, such as a micro RNA (miRNA), small interfering RNA (siRNA) or short hairpin RNA (shRNA). Hence, in accordance with a preferred embodiment, the nucleic acid molecule is a micro RNA. In accordance with another preferred embodiment, the nucleic acid molecule is a small interfering RNA. In accordance with another preferred embodiment, the nucleic acid molecule is a short hairpin RNA.

As a result, an engineered T-cell is obtained which is characterized in that the expression of CIITA is inhibited. It is also contemplated by the present invention that the engineered T-cell of the present invention does not express a functional T-cell receptor (TCR) on its cell surface. T-cell receptors are cell surface receptors that participate in the activation of T cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V (D) J recombination, creating a large diversity of antigen specificities within the population of T cells. However, in contrast to immunoglobulins that recognize intact antigen, T-cells are activated by processed peptide fragments in association with an MHC molecule, introducing an extra dimension to antigen recognition by T cells, known as MHC restriction. Recognition of MHC disparities between the donor and recipient through the T-cell receptor leads to T-cell proliferation and the potential development of graft versus host disease (GVHD). It has been shown that normal surface expression of the TCR depends on the coordinated synthesis and assembly of all seven components of the complex (Ashwell and Klusner 1990). The inactivation of TCR alpha or TCR beta can result in the elimination of the TCR from the surface of T-cells preventing recognition of alloantigen and thus GVHD. The inactivation of at least one gene coding for a TCR component thus renders the engineered T-cell less alloreactive. By “inactivating” or “inactivation of” a gene it is meant that the gene of interest (e.g., at least one gene coding for a TCR component) is not expressed in a functional protein form.

Therefore, the method of the present invention in accordance with particular embodiments further comprises inactivating at least one gene encoding a component of the T-cell receptor. More particularly, the inactivation is achieved by using (e.g., introducing into the T-cell) a rare-cutting endonuclease able to selectively inactivate by DNA cleavage, preferably double-strand break, at least one gene encoding a component of the T-Cell receptor (TCR). According to particular embodiments, the rare-cutting endonuclease is able to selectively inactivate by DNA cleavage the gene coding for TCR alpha or TCR beta. According to a preferred embodiment, the rare-cutting endonuclease is able to selectively inactivate by DNA cleavage the gene coding for TCR alpha. Especially in case of an allogeneic T-cell obtained from a donor, inactivating of at least one gene encoding a component of TCR, notably TCR alpha, leads to engineered T-cells, when infused into an allogeneic host, which are non-alloreactive. This makes the engineered T-cell particular suitable for allogeneic transplantations, especially because it reduces the risk of graft versus host disease.

A rare-cutting endonuclease to be used in accordance of the present invention to inactivate at least one gene encoding a component of the T-cell receptor may, for instance, be a TAL-nuclease, meganuclease, zing-finger nuclease (ZFN), or RNA guided endonuclease (such as Cas9).

According to a particular embodiment, the rare-cutting endonuclease is a TAL-nuclease.

According to another particular embodiment, the rate-cutting endonuclease is a homing endonuclease, also known under the name of meganuclease.

According to another particular embodiment, the rare-cutting endonuclease is a zing-finger nuclease (ZNF).

According to another particular embodiment, the rare-cutting endonuclease is a RNA guided endonuclease. According to a preferred embodiment, the RNA guided endonuclease is the Cas9/CRISPR complex.

In order to be expressed in the T-cell, said rare-cutting endonuclease may be introduced into the cell by way of an exogenous nucleic acid molecule comprising a nucleotide sequence encoding said rare-cutting endonuclease. According to particular embodiments, the method of the invention further comprises introducing into said T-cell an exogenous nucleic acid molecule comprising a nucleotide sequence coding for a rare-cutting endonuclease able to selectively inactivate by DNA cleavage, preferably double-strand break, at least one gene encoding a component of the T-cell receptor (TCR).

As a result, an engineered T-cell is obtained which further expresses a rare-cutting endonuclease able to selectively inactivate by DNA cleavage at least one gene encoding a component of the T-cell receptor (TCR). In consequence, an engineered T-cell is obtained which is characterized in that at least at least one gene encoding a component of the T-cell receptor (TCR) is inactivated.

It is also contemplated by the present invention that the engineered T-cell further expresses a Chimeric Antigen Receptor (CAR) directed against at least one antigen expressed at the surface of a malignant or infected cell. Hence, in accordance with certain embodiments, the method of the invention furthers comprise introducing into said T-cell an exogenous nucleic acid molecule comprising a nucleotide sequence coding for a Chimeric Antigen Receptor (CAR) directed against at least one antigen expressed at the surface of a malignant or infected cell.

The T-cell to be modified according to the present invention may be any suitable T-cell. For example, the T-cell can be an inflammatory T-lymphocyte, cytotoxic T-lymphocyte, regulatory T-cell or helper T-lymphocyte. Particularly, the T-cell is a cytotoxic T-lymphocyte. In certain embodiments, said T-cell is selected from CD4+T-lymphocytes and CD8+T-lymphocytes. They can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. In particular embodiments, the T-cell to be modified according to the present invention is a human T-cell. Prior to expansion and genetic modification of the cells of the invention, a source of cells can be obtained from a subject, such as a patient, through a variety of non-limiting methods. T-cell can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics.

### Rare-Cutting Endonuclease

In accordance with certain embodiments of the present invention, rare-cutting endonucleases are employed which are able to selectively inactivate by DNA cleavage the gene of interest, such as the gene encoding B2M.

The term “rare-cutting endonuclease” refers to a wild type or variant enzyme capable of catalyzing the hydrolysis (cleavage) of bonds between nucleic acids within a DNA or RNA molecule, preferably a DNA molecule. Particularly, said nuclease can be an endonuclease, more preferably a rare-cutting endonuclease which is highly specific, recognizing nucleic acid target sites ranging from 10 to 45 base pairs (bp) in length, usually ranging from 10 to 35 base pairs in length, more usually from 12 to 20 base pairs. The endonuclease according to the present invention recognizes at specific polynucleotide sequences, further referred to as “target sequence” and cleaves nucleic acid inside these target sequences or into sequences adjacent thereto, depending on the molecular structure of said endonuclease. The rare-cutting endonuclease can recognize and generate a single- or double-strand break at specific polynucleotides sequences.

In particular embodiments, said rare-cutting endonuclease according to the present invention is a RNA-guided endonuclease such as the Cas9/CRISPR complex. RNA guided endonucleases constitute a new generation of genome engineering tool where an endonuclease associates with a RNA molecule. In this system, the RNA molecule nucleotide sequence determines the target specificity and activates the endonuclease. Cas9, also named Csn1 is a large protein that participates in both crRNA biogenesis and in the destruction of invading DNA. Cas9 has been described in different bacterial species such as S. thermophiles, Listeria innocua and S. Pyogenes. The large Cas9 protein (>1200 amino acids) contains two predicted nuclease domains, namely HNH (McrA-like) nuclease domain that is located in the middle of the protein and a splitted RuvC-like nuclease domain (RNase H fold). Cas9 variant can be a Cas9 endonuclease that does not naturally exist in nature and that is obtained by protein engineering or by random mutagenesis. Cas9 variants according to the invention can for example be obtained by mutations i.e. deletions from, or insertions or substitutions of at least one residue in the amino acid sequence of a S. pyogenes Cas9 endonuclease (COG3513).

In other particular embodiments, said rare-cutting endonuclease can also be a homing endonuclease, also known under the name of meganuclease. Such homing endonucleases are well-known to the art. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-Crel variant. A “variant” endonuclease, i.e. an endonuclease that does not naturally exist in nature and that is obtained by genetic engineering or by random mutagenesis can bind DNA sequences different from that recognized by wild-type endonucleases (see international application WO2006/097854).

In other particular embodiments, said rare-cutting endonuclease can be a “Zinc Finger Nucleases” (ZFNs), which are generally a fusion between the cleavage domain of the type IIS restriction enzyme, Fokl, and a DNA recognition domain containing 3 or more C2H2 zinc finger motifs. The heterodimerization at a particular position in the DNA of two individual ZFNs in precise orientation and spacing leads to a double-strand break (DSB) in the DNA. The use of such chimeric endonucleases have been extensively reported in the art as reviewed by Urnov et al. (Genome editing with engineered zinc finger nucleases (2010) Nature reviews Genetics 11:636-646). Standard ZFNs fuse the cleavage domain to the C-terminus of each zinc finger domain. In order to allow the two cleavage domains to dimerize and cleave DNA, the two individual ZFNs bind opposite strands of DNA with their C-termini a certain distance apart. The most commonly used linker sequences between the zinc finger domain and the cleavage domain requires the 5′ edge of each binding site to be separated by 5 to 7 bp. The most straightforward method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells.

In other particular embodiments, said rare-cutting endonuclease is a “TALE-nuclease” or a “MBBBD-nuclease” resulting from the fusion of a DNA binding domain typically derived from Transcription Activator Like Effector proteins (TALE) or from a Modular Base-per-Base Binding domain (MBBBD), with a catalytic domain having endonuclease activity. Such catalytic domain usually comes from enzymes, such as for instance I-Tevl, ColE7, NucA and Fok-I. TALE-nuclease can be formed under monomeric or dimeric forms depending of the selected catalytic domain (WO2012138927). Such engineered TALE-nucleases are commercially available under the trade name TALENT (Cellectis, 8 rue de la Croix Jarry, 75013 Paris, France). In general, the DNA binding domain is derived from a Transcription Activator like Effector (TALE), wherein sequence specificity is driven by a series of 33-35 amino acids repeats originating from Xanthomonas or Ralstonia bacterial proteins AvrBs3, PthXo1, AvrHah1, PthA, Tal1c as non-limiting examples. These repeats differ essentially by two amino acids positions that specify an interaction with a base pair. Each base pair in the DNA target is contacted by a single repeat, with the specificity resulting from the two variant amino acids of the repeat (the so-called repeat variable dipeptide, RVD). TALE binding domains may further comprise an N-terminal translocation domain responsible for the requirement of a first thymine base (TO) of the targeted sequence and a C-terminal domain that containing a nuclear localization signals (NLS). A TALE nucleic acid binding domain generally corresponds to an engineered core TALE scaffold comprising a plurality of TALE repeat sequences, each repeat comprising a RVD specific to each nucleotides base of a TALE recognition site. In the present invention, each TALE repeat sequence of said core scaffold is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A. In another embodiment, critical amino acids 12 and 13 can be mutated towards other amino acid residues in order to modulate their specificity towards nucleotides A, T, C and G and in particular to enhance this specificity. A TALE nucleic acid binding domain usually comprises between 8 and 30 TALE repeat sequences. More preferably, said core scaffold of the present invention comprises between 8 and 20 TALE repeat sequences; again more preferably 15 TALE repeat sequences. It can also comprise an additional single truncated TALE repeat sequence made of 20 amino acids located at the C-terminus of said set of TALE repeat sequences, i.e. an additional C-terminal half-TALE repeat sequence. Other modular base-per-base specific nucleic acid binding domains (MBBBD) are described in WO 2014/018601. Said MBBBD can be engineered, for instance, from newly identified proteins, namely EAV36_BURRH, E5AW43_BURRH, E5AW45_BURRH and E5AW46_BURRH proteins from the recently sequenced genome of the endosymbiont fungi Burkholderia Rhizoxinica. These nucleic acid binding polypeptides comprise modules of about 31 to 33 amino acids that are base specific. These modules display less than 40% sequence identity with Xanthomonas TALE common repeats and present more polypeptides sequence variability. The different domains from the above proteins (modules, N and C-terminals) from Burkholderia and Xanthomonas are useful to engineer new proteins or scaffolds having binding properties to specific nucleic acid sequences and may be combined to form chimeric TALE-MBBBD proteins.

### Inhibitory Nucleic Acid Molecules

In accordance with certain other embodiments of the present invention, nucleic acid molecules are employed which inhibit the expression of B2M. More particularly, the nucleic acid may be an antisense oligonucleotide, ribozyme or interfering RNA (RNAi) molecule. Preferably, such nucleic acid molecule comprises at least 10 consecutive nucleotides of the complement of SEQ ID NO: 3.

According to particular embodiments, the inhibitory nucleic acid is an antisense oligonucleotide which inhibits the expression of B2M. Such antisense oligonucleotide is an nucleic acid (either DNA or RNA) which specifically hybridizes (e.g. binds) under cellular conditions with the cellular mRNA and/or genomic DNA encoding B2M, thereby inhibiting transcription and/or translation of the gene. The binding may be by conventional base pair complementarity. Alternatively, the binding may be, for example, in case of binding to DNA duplexes, through specific interactions in the major groove of the double helix. Absolute complementarity, although preferred, is not required.

Also contemplated by the present invention is that nucleic acid molecules are employed which inhibit the expression of CIITA. More particularly, the nucleic acid may be an antisense oligonucleotide, ribozyme or interfering RNA (RNAi) molecule. Preferably, such nucleic acid molecule comprises at least 10 consecutive nucleotides of the complement of SEQ ID NO: 5.

Antisense oligonucleotides employed according to the invention may be DNA or RNA or chimeric mixtures or derivatives or modified versions thereof, and may be single-stranded or double stranded. Thus, according to a preferred embodiment, the antisense oligonucleotide is a single-stranded or double-stranded DNA molecule, more preferably a double-stranded DNA molecule. According to another preferred embodiment, the antisense oligonucleotide is a single-stranded or double-stranded RNA molecule, more preferably a single-stranded RNA molecule.

According to preferred embodiments, the antisense oligonucleotide is a modified oligonucleotide which is resistant to endogenous nucleases, e.g., exonucleases and/or endonucleases, and is therefore stable in vivo and in vitro.

The antisense oligonucleotide may be modified at the base moiety, sugar moiety, or phosphate backbone, for example, to improve stability of the molecule. The antisense oligonucleotide may include other appended groups such as peptides (e.g., for targeting host cell receptors), or agents facilitating transport across the cell membrane. Hence, the antisense oligonucleotide may be conjugated to another molecule such as a peptide or transport agent.

According to particular embodiments, the antisense oligonucleotide comprises at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w and 2,6-diaminopurine.

According to other particular embodiments, the antisense oligonucleotide comprise at least one modified sugar moiety selected from the group including, but not limited to, arabinose, 2-fluoroarabinose, xylulose and hexose.

According to other particular embodiments, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

An antisense oligonucleotide may be delivered into the cell, for example, in form of an expression vector, such as a plasmid or viral vector, which, when transcribed in the cells, produces RNA which is complementary to at least a unique portion of the cellular mRNA for B2M. Alternatively, the antisense oligonucleotide may be generated ex vivo and introduced into the cell by any known means in the art. The antisense oligonucleotide may be synthesise ex vivo by standard method known in the art, e.g., by use of an automated DNA synthesizer (such as automated DNA synthesizer are commercially available from, e.g., Applied Biosystems). A number of methods have been developed for delivering antisense DNA or RNA to cells, e.g. by direct injection or through modification designed to target the desired cell (e.g., using antisense oligonucleotides linked to peptides or antibodies that specifically bind receptors or antigens expressed on the target cell surface.

According to preferred embodiments, a recombinant DNA vector is used in which a nucleotide sequence coding for an antisense oligonucleotide inhibiting the expression of B2M or CIITA is placed under the control of a promoter, such as a strong pol Ill or pol II promoter. The use of such a construct to transfect a target cell, such as a T-cell, will result in the transcription of a sufficient amount of single-stranded RNA that will form complementary base pairs with the endogenous transcript and thereby prevent translation of the B2M or CIITA mRNA. In accordance with these embodiments, a DNA vector comprising the nucleotide sequence encoding the antisense oligonucleotide is introduced into the cell where the transcription of an antisense RNA occurs. Such vector can remain episomal or be chromosomally integrated, as long as it can be transcribed to produce the antisense RNA. The expression of the sequence encoding the antisense RNA can be by any promoter known in the art to act in mammalian, preferably human cells. Such promoter can be inducible or constitutive. Exemplary promoters include, but are not limited to, the SV40 early promoter region, the promoter containing the 3′ long terminal repeat of Rous sarcoma virus, the herpes thymidine promoter, and the regulatory sequences of the methallothionein gene.

Alternatively, antisense cDNA constructs that synthesize antisense RNA constitutively or inducibly, depending on the promoter used, can be introduced into the cell.

According to preferred embodiments, the antisense oligonucleotide comprises at least 10 consecutive nucleotides of the complement of SEQ ID NO: 3. In case of a double stranded molecule, such double-stranded antisense oligonucleotide comprises a first strand comprising at least 10 consecutive nucleotide of SEQ ID NO: 3, and a second strand complementary to said first strand. In case of a single-stranded molecule, such single-stranded oligonucleotide comprises at least 10 consecutive nucleotides of the complement of SEQ ID NO: 3.

According to other preferred embodiments, the antisense oligonucleotide comprises at least 10 consecutive nucleotides of the complement of SEQ ID NO: 5. In case of a double stranded molecule, such double-stranded antisense oligonucleotide comprises a first strand comprising at least 10 consecutive nucleotide of SEQ ID NO: 5, and a second strand complementary to said first strand. In case of a single-stranded molecule, such single-stranded oligonucleotide comprises at least 10 consecutive nucleotides of the complement of SEQ ID NO: 5.

The antisense oligonucleotide may comprise a nucleotide sequence complementary to a non-coding or a coding region of the B2M or CIITA mRNA. According to preferred embodiments, the antisense oligonucleotide comprises a nucleotide sequence complementary to the 5′ end of the B2M or CIITA mRNA, e.g., the 5′ untranslated sequence up to and including the AUG initiation codon. According to other preferred embodiments, the antisense oligonucleotide comprises a nucleotide sequence complementary to the 3′ untranslated sequence of the B2M or CIITA mRNA. According to other preferred embodiments, the antisense oligonucleotide comprises a nucleotide sequence complementary to the coding region of the B2M or CIITA mRNA. Whether designed to hybridize to the 5′, 3′ or coding region of the B2M or CIITA mRNA, an antisense oligonucleotide should be at least six nucleotides in length, preferably at least 10 nucleotide in length, and is preferably less than about 100, and more preferably less than about 50, 25, 20, 15 or 10 nucleotides in length. According to preferred embodiments, the antisense oligonucleotide is 6 to 25, such as 10 to 25 nucleotides in length.

In accordance with other particular embodiments, a ribozyme molecule designed to catalytically cleave the B2M or CIITA mRNA transcript is used to prevent translation and expression of B2M or CIITA in the T-cell, respectively (see, e.g., WO 90/11364 and U.S. Pat. No. 5,093,246 for general guidance). According to preferred embodiments, the ribozyme is a hammerhead ribozyme. Hammerhead ribozymes cleave mRNAs at locations dictated by flanking regions that form complementary base pairs with the target mRNA, e.g. the B2M mRNA, such as the human B2M mRNA set forth in SEQ ID NO: 3. The sole requirement is that the target mRNA has the following sequence of two bases: 5′-UG-3′. The constructions and production of hammerhead ribozymes is well known in the art and is described in more detail in Haseloff and Gerlach (1988). In accordance with preferred embodiments, the ribozyme is engineered such that the cleavage recognition site is located near the 5′ end of the B2M mRNA. In accordance with preferred other embodiments, the ribozyme is engineered such that the cleavage recognition site is located near the 5′ end of the CIITA mRNA. This increases the efficiency and minimizes the intracellular accumulation of non-functional mRNA transcripts.

Like with antisense oligonucleotides, a riboyzme used in accordance with the invention may be composed of modified oligonucleotides to, e.g., improve stability. The ribozyme may be delivered to the cell by any means known in the art. The ribozyme may be delivered to the T-cell in form of an expression vector, such as a plasmid or viral vector, which, when transcribed in the cells, produces the ribozyme. According to preferred embodiments, a recombinant DNA vector is used in which a nucleotide sequence coding for the ribozyme is placed under the control of a promoter, such as a strong pol III or pol II promoter, so that a transfected cell will produce sufficient amounts of the ribozyme to destroy endogenous mRNA and inhibit translation. Because riboyzmes, unlike antisense oligonucleotides, are cataylitc, a lower intracellular concentration is required for efficiency.

In accordance with other particular embodiments, the inhibitory nucleic acid is an interfering RNA (RNAi) molecule. RNA interference is a biological process in which RNA molecules inhibit gene expression, typically causing the destruction of specific mRNA. Exemplary types of RNAi molecules include microRNA (miRNA), small interfering RNA (siRNA) and short hairpin RNA (shRNA). According to a preferred embodiment, the RNAi molecule is a miRNA. According to another preferred embodiment, the RNAi molecule is a siRNA. According to yet another preferred embodiment, the RNAi molecule is a shRNA. The production of RNAi molecules in vivo and in vitro and their methods of use are described in, e.g., U.S. Pat. No. 6,506,559, WO 01/36646, WO 00/44895, US2002/01621126, US2002/0086356, US2003/0108923, WO 02/44321, WO 02/055693, WO 02/055692 and WO 03/006477.

In accordance with a preferred embodiment, the RNAi molecule is an interfering RNA complementary to SEQ ID NO: 3. In accordance to another preferred embodiment, the RNAi molecule is a ribonucleic acid molecule comprising at least 10 consecutive nucleotides of the complement of SEQ ID NO: 3. In accordance with another preferred embodiment, the RNAi molecule is a double-stranded ribonucleic acid molecule comprising a first strand identical to 20 to 25, such as 21 to 23, consecutive nucleotides of SEQ ID NO: 3, and a second strand complementary to said first strand.

In accordance with a preferred embodiment, the RNAi molecule is an interfering RNA complementary to SEQ ID NO: 5. In accordance to another preferred embodiment, the RNAi molecule is a ribonucleic acid molecule comprising at least 10 consecutive nucleotides of the complement of SEQ ID NO: 5. In accordance with another preferred embodiment, the RNAi molecule is a double-stranded ribonucleic acid molecule comprising a first strand identical to 20 to 25, such as 21 to 23, consecutive nucleotides of SEQ ID NO: 5, and a second strand complementary to said first strand.

### Engineering of the PD1/PDL1 Pathway of T-Cell Regulation

The present invention aims at facilitating the engraftment of T-cells, especially allogeneic T-cells, preferably by inhibiting the expression of B2M and/or CIITA in combination with inactivation of TCR.

As an alternative to or in combination with this approach, the inventors have found that T-cells can be disrupted for PD1 (Programmed cell death protein 1, also known as PD1; PD-1; CD279; SLEB2; hPD-1; hPD-I or hSLE1), which is a 288 amino acid cell surface protein molecule encoded by the PDCD1 gene (NCBI-NC_000002.12). This protein is expressed on T cells and pro-B cells and has been found to negatively regulate T-cell responses (Carter L., et al., 2002). The formation of PD-1 receptor/PD-L1 ligand complex transmits an inhibitory signal, which reduces the proliferation of T-cells.

Programmed death ligand 1 (PD-L1) is a 40 kDa type 1 transmembrane protein that is deemed to play a major role in suppressing the immune system during particular events such as pregnancy, tissue allografts, autoimmune disease and other disease states such as hepatitis. PDL-1 (also called CD274 or B7H1) is encoded by CD274 gene (NCBI-NM_014143).

According to a particular aspect, the expression of both PD-1 and TCR are inhibited in the engineered T-cells of the invention, which has the dual effect of activating the T-cells as part of an allogeneic transplantation. However, the inactivation or inhibition of PD-1 can be also implemented as part of an autologous transplantation of T-cells, where the inhibition or disruption of TCR would not be required.

According to a further aspect of the invention, the inhibition or disruption of PD1 is combined with the over-expression of its ligand PDL-1 in the transplanted T-cells. This over-expression can be obtained, for instance, upon lentiviral or retroviral transformation in T-cells, in which PD-1 is inhibited or disrupted, or by any other means reported in the art. Accordingly, PDL1 that is over-expressed by the T-cells will not affect the [PD1⁻] transplanted cells, but only the [PD1⁺] T-cells from the patient. As a result, the T-cells from the patient are inhibited and do not activate against the transplanted cells, which facilitates their engraftment and persistence into the host.

According to a preferred embodiment, the invention provides engineered T-cells which are [PD1⁻]⁻[TCR⁻], while overexpressing PDL1 to facilitate their transplantation into a patient, in particular as part of an immunotherapy.

### Expression of at Least One Non-Endogenous Immunosuppressive Polypeptide

According to some preferred embodiments, the inhibition of the expression of the beta-2m and/or the CIITA is carried out with an additional step of expression in said T-cell of at least one non-endogenous immunosuppressive polypeptide.

By “non-endogenous” polypeptide is meant a polypeptide not normally expressed by a donor's immune cell, preferably a polypeptide expressed by an exogenous polynucleotide that has been imported into the immune's cell genome. For instance, IL12 is not considered hereby as being a non-endogenous polypeptide because it is expressed from a preexisting gene from the donor's immune cell.

By “immunosuppressive” is meant that the expression of said non-endogenous polypeptide has the effect of alleviating the immune response of the patient host against the donor's immune cells.

The method of the present invention may thus comprise introducing into said T-cell an exogenous nucleic acid molecule comprising a nucleotide sequence coding for at least one non-endogenous immunosuppressive polypeptide, such as a viral MHC homolog or an NKG2D ligand.

### Expression of Viral MHC Homolog

According to particularly preferred embodiments, said non-endogenous immunosuppressive polypeptide expressed in said T-cell is a viral MHC homolog, such as for instance UL18 (referred to as NP_044619 in the NCBI protein database).

According to these embodiments, the method of the present invention may thus comprise introducing into said T-cell an exogenous nucleic acid molecule comprising a nucleotide sequence coding for a viral MHC homolog, such as UL18. The exogenous nucleic acid molecule may comprise a nucleotide sequence coding for a polypeptide sharing at least 80%, preferably at least 90% and more preferably at least 95% of identity with SEQ ID NO: 89.

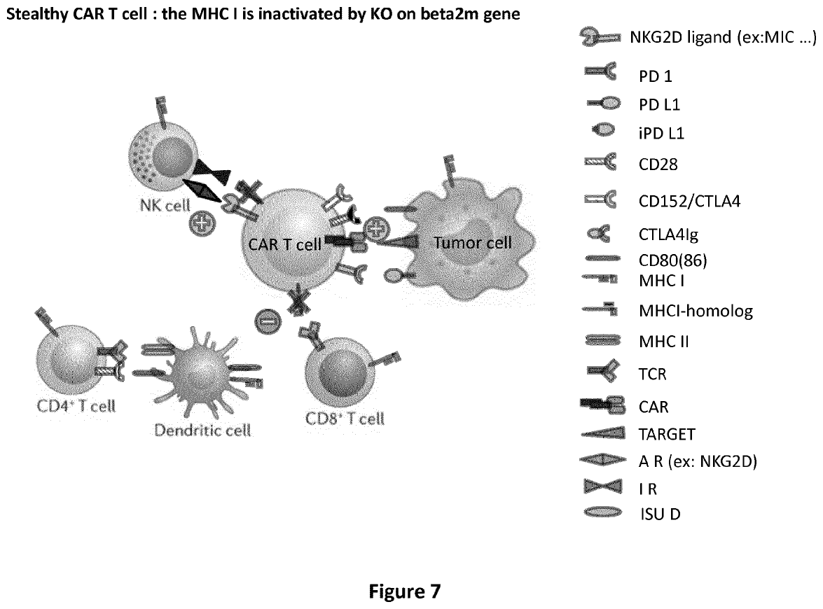

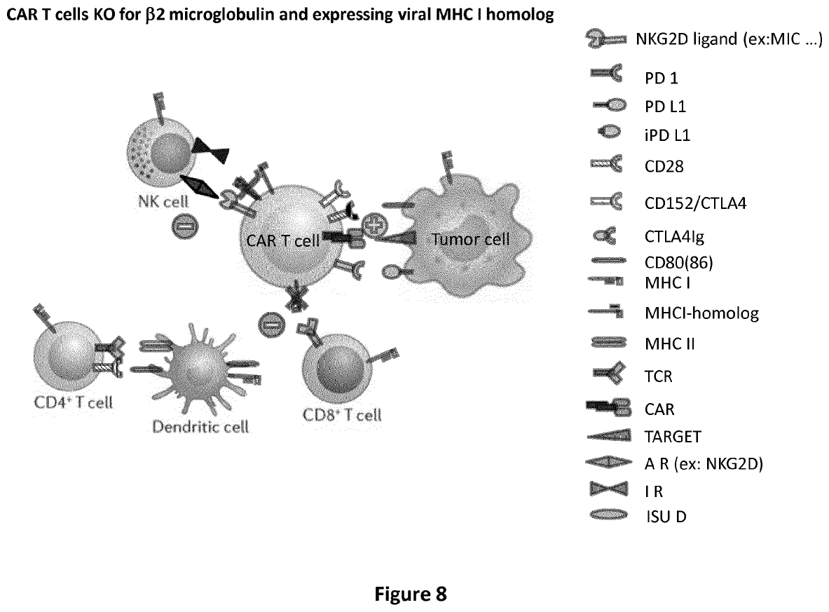

The interaction between the allogeneic T cell and host immune cells is schematically represented in FIG. 8 (expression of viral MHC homolog) in regard to the situation to FIG. 7 (no expression). In both figures, the MHC class I is preferably inactivated by disrupting (KO) the beta2M gene.

### Expression of NKG2D Ligand

Some viruses such as cytomegaloviruses have acquired mechanisms to avoid NK cell mediate immune surveillance and interfere with the NKG2D pathway by secreting a protein able to bind NKG2D ligands and prevent their surface expression (Welte, S. A.; Sinzger, C.; Lutz, S. Z.; Singh-Jasuja, H.; Sampaio, K. L.; Eknigk, U.; Rammensee, H. G.; Steinle, A. 2003 “Selective intracellular retention of virally induced NKG2D ligands by the human cytomegalovirus UL16 glycoprotein”. Eur. J. Immunol., 33, 194-203). In tumors cells, some mechanisms have evolved to evade NKG2D response by secreting NKG2D ligands such as ULBP2, MICB or MICA (Salih H R, Antropius H, Gieseke F, Lutz S Z, Kanz L, et al. (2003) Functional expression and release of ligands for the activating immunoreceptor NKG2D in leukemia. Blood 102:1389-1396)

According to other particularly preferred embodiments, the non-endogenous immunosuppressive polypeptide to be expressed in said T-cell is an NKG2D ligand.

According to these embodiments, the method of the present invention may thus comprise introducing into said T-cell an exogenous nucleic acid molecule comprising a nucleotide sequence coding for an NKG2D ligand. The nucleic acid molecule may comprise a nucleotide sequence coding for a polypeptide sharing at least 80%, preferably at least 90% and more preferably at least 95% of identity with any one of SEQ ID NO: 90-97.

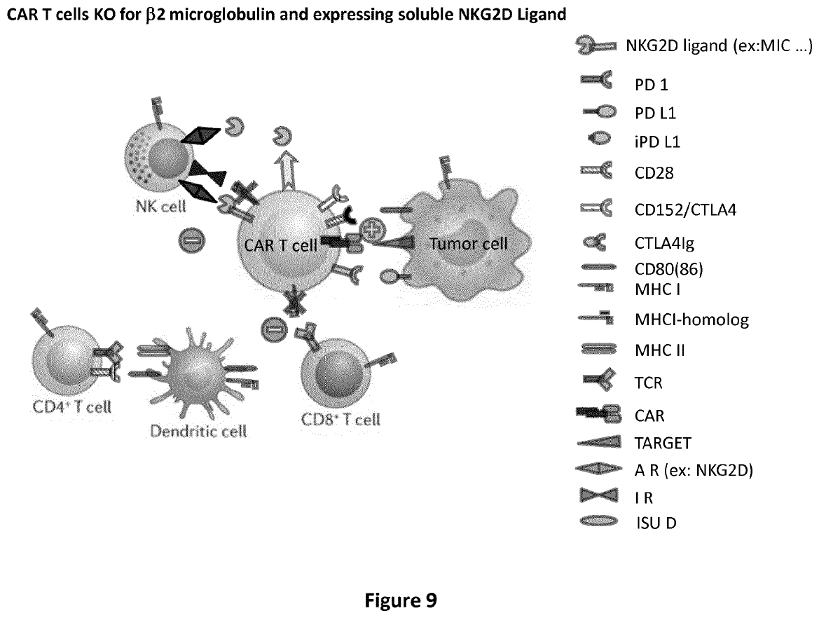

The interaction between the allogeneic T cell and host immune cells is schematically represented in FIG. 9 (expression of soluble NKG2D ligand) in regard to the situation to FIG. 7 (no expression). In both figures, the MHC class I is inactivated by disrupting (KO) the beta2M gene.

The Table 10 presented further in the text represents a viral MHC homolog (UL18) and a panel of NKG2D ligands and their polypeptide sequence to be expressed according to the present invention.

### Chimeric Antigen Receptors (CARs)

Adoptive immunotherapy, which involves the transfer of autologous antigen-specific T-cells generated ex vivo, is a promising strategy to treat cancer or viral infections. The T-cells used for adoptive immunotherapy can be generated either by expansion of antigen-specific T cells or redirection of T cells through genetic engineering. Transfer of viral antigen specific T-cells is a well-established procedure used for the treatment of transplant associated viral infections and rare viral-related malignancies. Similarly, isolation and transfer of tumor specific T cells has been shown to be successful in treating melanoma. Novel specificities in T-cells have been successfully generated through the genetic transfer of transgenic T-cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T-cells. CARs have successfully allowed T-cells to be redirected against antigens expressed at the surface of tumor cells from various malignancies including lymphomas and solid tumors.

CD19 is an attractive target for immunotherapy because the vast majority of B-acute lymphoblastic leukemia (B-ALL) uniformly express CD19, whereas expression is absent on non hematopoietic cells, as well as myeloid, erythroid, and T cells, and bone marrow stem cells. Clinical trials targeting CD19 on B-cell malignancies are underway with encouraging anti-tumor responses. Most infuse T cells genetically modified to express a chimeric antigen receptor (CAR) with specificity derived from the scFv region of a CD19-specific mouse monoclonal antibody FMC63 (WO2013/126712).

Therefore, in accordance with certain embodiments, the Chimeric Antigen Receptor expressed by the engineered T-cell is directed against the B-lymphocyte antigen CD19.

In accordance with certain embodiments, the Chimeric Antigen Receptor is a single chain Chimeric Antigen Receptor. As an example of single-chain Chimeric Antigen Receptor to be expressed in the engineered T-cells according to the present invention is a single polypeptide that comprises at least one extracellular ligand binding domain, a transmembrane domain and at least one signal transducing domain, wherein said extracellular ligand binding domain comprises a scFV derived from the specific anti-CD19 monoclonal antibody 4G7. Once transduced into the T-cell, for instance by using retroviral or lentiviral transduction, this CAR contributes to the recognition of CD19 antigen present at the surface of malignant B-cells involved in lymphoma or leukemia.

In accordance with particular embodiments, the Chimeric Antigen Receptor is a polypeptide comprising the amino acid sequence forth in SEQ ID NO: 6 or a variant thereof comprising an amino acid sequence that has at least 70%, such as at least 80%, at least 90%, at least 95%, or at least 99%, sequence identity with the amino acid sequence set forth in SEQ ID NO: 6 over the entire length of SEQ ID NO: 6. Preferably, the variant is capable of binding CD19.

A particularly preferred Chimeric Antigen Receptor is a polypeptide comprising the amino acid sequence set forth in SEQ ID NO: 7 or a variant thereof comprising an amino acid sequence that has at least 80%, such as at least 90%, at least 95%, or at least 99%, sequence identity with the amino acid sequence set forth in SEQ ID NO: 7 over the entire length of SEQ ID NO: 7. Such variant may differ from the polypeptide set forth in SEQ ID NO: 7 in the substitution of at least one, at least two or at least three amino acid residue(s). Preferably, said variant is capable of binding CD19.

In accordance with other certain embodiments, the Chimeric Antigen Receptor may be directed against another antigen expressed at the surface of a malignant or infected cell, such as a cluster of differentiation molecule, such as CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), multiple myeloma or lymphoblastic leukaemia antigen, such as one selected from TNFRSF17 (UNIPROT Q02223), SLAMF7 (UNIPROT Q9NQ25), GPRC5D (UNIPROT Q9NZD1), FKBP11 (UNIPROT Q9NYL4), KAMP3, ITGA8 (UNIPROT P53708), and FCRL5 (UNIPROT Q68SN8). a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface antigens.

In other certain embodiments, the Chimeric Antigen Receptor is a multi-chain Chimeric Antigen Receptor. Chimeric Antigen Receptors from the prior art introduced in T-cells have been formed of single chain polypeptides that necessitate serial appending of signaling domains.

However, by moving signaling domains from their natural juxtamembrane position may interfere with their function. To overcome this drawback, the applicant recently designed a multi-chain CAR derived from FcERI to allow normal juxtamembrane position of all relevant signaling domains. In this new architecture, the high affinity IgE binding domain of FcERI alpha chain is replaced by an extracellular ligand-binding domain such as scFv to redirect T-cell specificity against cell targets and the N and/or C-termini tails of FcERI beta chain are used to place costimulatory signals in normal juxtamembrane positions as described in WO 2013/176916.

Accordingly, a CAR expressed by the engineered T-cell according to the invention can be a multi-chain chimeric antigen receptor particularly adapted to the production and expansion of engineered T-cells of the present invention. Such multi-chain CARs comprise at least two of the following components:
a) one polypeptide comprising the transmembrembrane domain of FcERI alpha chain and an extracellular ligand-binding domain,
b) one polypeptide comprising a part of N- and C-terminal cytoplasmic tail and the transmembrane domain of FcERI beta chain and/or
c) at least two polypeptides comprising each a part of intracytoplasmic tail and the transmembrane domain of FcERI gamma chain, whereby different polypeptides multimerize together spontaneously to form dimeric, trimeric or tetrameric CAR.

According to such architectures, ligands binding domains and signaling domains are born on separate polypeptides. The different polypeptides are anchored into the membrane in a close proximity allowing interactions with each other. In such architectures, the signaling and co-stimulatory domains can be in juxtamembrane positions (i.e. adjacent to the cell membrane on the internal side of it), which is deemed to allow improved function of co-stimulatory domains. The multi-subunit architecture also offers more flexibility and possibilities of designing CARs with more control on T-cell activation. For instance, it is possible to include several extracellular antigen recognition domains having different specificity to obtain a multi-specific CAR architecture. It is also possible to control the relative ratio between the different subunits into the multi-chain CAR. This type of architecture has been recently detailed by the applicant in PCT/US2013/058005.

The assembly of the different chains as part of a single multi-chain CAR is made possible, for instance, by using the different alpha, beta and gamma chains of the high affinity receptor for IgE (FcERI) to which are fused the signaling and co-stimulatory domains. The gamma chain comprises a transmembrane region and cytoplasmic tail containing one immunoreceptor tyrosine-based activation motif (ITAM).

The multi-chain CAR can comprise several extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the multi-chain CAR.

The signal transducing domain or intracellular signaling domain of the multi-chain CAR(s) of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the multi-chain CAR is expressed. For example, the effector function of a T cell can be a cytolytic activity or helper activity including the secretion of cytokines.

In the present application, the term “signal transducing domain” refers to the portion of a protein which transduces the effector signal function signal and directs the cell to perform a specialized function.

Preferred examples of signal transducing domain for use in single or multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. According to particular embodiments, the signaling transducing domain of the multi-chain CAR can comprise the CD3zeta signaling domain, or the intracytoplasmic domain of the FcERI beta or gamma chains.

According to particular embodiments, the signal transduction domain of multi-chain CARs of the present invention comprises a co-stimulatory signal molecule. A co-stimulatory molecule is a cell surface molecule other than an antigen receptor or their ligands that is required for an efficient immune response.

Ligand binding-domains can be any antigen receptor previously used, and referred to, with respect to single-chain CAR referred to in the literature, in particular scFv from monoclonal antibodies.

### Engineered T-Cells

As a result of the present invention, engineered T-cells can be obtained having improved characteristics. In particular, the present invention provides an engineered, preferably isolated, T-cell which is characterized in that the expression of B2M and/or CIITA is inhibited.

According to certain embodiments, the present invention provides an engineered, preferably isolated, T-cell which expresses a rare-cutting endonuclease able to selectively inactivate by DNA cleavage, preferably double-strand break, the gene encoding B2M. According to particular embodiments, said T-cell comprises an exogenous nucleic acid molecule comprising a nucleotide sequence encoding said rare-cutting endonuclease. According to more particular embodiments, said rare-cutting endonuclease is a TAL-nuclease, meganuclease, zinc-finger nuclease (ZFN), or RNA guided endonuclease. Hence, in accordance with a specific embodiment, the rare-cutting endonuclease is a TAL-nuclease. In accordance with another specific embodiment, the rare-cutting endonuclease is a meganuclease. In accordance with another specific embodiment, the rare-cutting endonuclease is a zinc-finger nuclease. In accordance with yet another specific embodiment, the rare-cutting endonuclease is a RNA guided endonuclease, such as Cas9.

According to certain other embodiments, the present invention provides an engineered, preferably isolated, T-cell which comprises an exogenous nucleic acid molecule that inhibits the expression of B2M. According to particular embodiments, said T-cell comprises an exogenous nucleic acid molecule comprising a nucleotide sequence encoding a nucleic acid molecule that inhibits the expression of B2M. According to more particular embodiments, the nucleic acid molecule that inhibits the expression of B2M is an antisense oligonucleotide, ribozyme or interfering RNA (RNAi) molecule. Hence, in accordance with a specific embodiment, nucleic acid molecule that inhibits the expression of B2M is an antisense oligonucleotide. In accordance with another specific embodiment, nucleic acid molecule that inhibits the expression of B2M is a ribozyme, and preferably a hammerhead riboyzme. In accordance with another specific embodiment, nucleic acid molecule that inhibits the expression of B2M is an interfering RNA molecule.

According to certain embodiments, the present invention provides an engineered, preferably isolated, T-cell which expresses a rare-cutting endonuclease able to selectively inactivate by DNA cleavage, preferably double-strand break, the gene encoding CIITA. According to particular embodiments, said T-cell comprises an exogenous nucleic acid molecule comprising a nucleotide sequence encoding said rare-cutting endonuclease. According to more particular embodiments, said rare-cutting endonuclease is a TAL-nuclease, meganuclease, zinc-finger nuclease (ZFN), or RNA guided endonuclease. Hence, in accordance with a specific embodiment, the rare-cutting endonuclease is a TAL-nuclease. In accordance with another specific embodiment, the rare-cutting endonuclease is a meganuclease. In accordance with another specific embodiment, the rare-cutting endonuclease is a zinc-finger nuclease. In accordance with yet another specific embodiment, the rare-cutting endonuclease is a RNA or DNA guided endonuclease, such as Cas9 or Argonaute.

According to certain other embodiments, the present invention provides an engineered, preferably isolated, T-cell which comprises an exogenous nucleic acid molecule that inhibits the expression of CIITA. According to particular embodiments, said T-cell comprises an exogenous nucleic acid molecule comprising a nucleotide sequence encoding a nucleic acid molecule that inhibits the expression of CIITA. According to more particular embodiments, the nucleic acid molecule that inhibits the expression of CIITA is an antisense oligonucleotide, ribozyme or interfereing RNA (RNAi) molecule. Hence, in accordance with a specific embodiment, nucleic acid molecule that inhibits the expression of CIITA is an antisense oligonucleotide. In accordance with another specific embodiment, nucleic acid molecule that inhibits the expression of CIITA is a ribozyme, and preferably a hammerhead riboyzme. In accordance with another specific embodiment, nucleic acid molecule that inhibits the expression of CIITA is an interfering RNA molecule.

According to certain embodiments, the engineered T-cell further expresses a rare-cutting endonuclease able to selectively inactivate by DNA cleavage, preferably double-strand break, at least one gene coding for a component of the T-cell receptor (TCR), such as TCR alpha. According to particular embodiments, said T-cell comprises an exogenous nucleic acid molecule comprising a nucleotide sequence encoding said rare-cutting endonuclease.

According to certain embodiments, the engineered T-cell further comprises expresses a Chimeric Antigen Receptor (CAR) directed against at least one antigen expressed at the surface of a malignant or infected cell. According to particular embodiments, said T-cell comprises an exogenous nucleic acid molecule comprising a nucleotide sequence encoding said CAR.

According to some embodiments, the present invention provides an engineered, preferably isolated, T-cell which expresses at least one non-endogenous immune-suppressive polypeptide. According to particular embodiments, said non-endogenous immune-suppressive polypeptide is a viral MHC homolog, such as UL18. The T-cell may thus comprise an exogenous nucleic acid molecule comprising a nucleotide sequence coding for a polypeptide sharing at least 80%, preferably at least 90% and more preferably at least 95% of identity with SEQ ID NO: 89. According to other particular embodiments, said non-endogenous immune-suppressive polypeptide is a NKG2D ligand. The T-cell may thus comprise an exogenous nucleic acid molecule comprising a nucleotide sequence coding for a polypeptide sharing at least 80%, preferably at least 90% and more preferably at least 95% of identity with any one of SEQ ID NO: 90-97.

It is understood that the details given herein in particularly with respect to the rare-cutting endonuclease able to selectively inactivate by DNA cleavage the gene encoding B2M, the nucleic acid molecule that inhibits the expression of B2M, the rare-cutting endonuclease able to selectively inactivate by DNA cleavage at least one gene coding for a component of the T-cell receptor (TCR), and the Chimeric Antigen Receptor also apply to this aspect of the invention.

Further, in the scope of the present invention is also encompassed a cell or cell line obtained from an engineered T-cell according to the invention, preferably displaying one of these phenotypes:
[b2m]⁻[TCR]⁻
[TCR]⁻[PD1]⁻[PDL-1]⁺
[b2m]⁻[TCR]⁻[PD1]⁻
[b2m]⁻[TCR]⁻[PD1]⁻[PDL-1]⁺
[b2m]⁻[viral MHC homolog]⁺
[b2m]⁻[TCR]⁻[viral MHC homolog]⁺
[b2m]⁻[NKG2D ligand]⁺
[b2m]⁻[TCR]⁻[NKG2D ligand]⁺

The T cells according to the present invention are preferably [CAR]⁺—i.e. armed with a chimeric antigen receptor to direct the specific recognition of tumor cells.

### Delivery Methods

The inventors have considered any means known in the art to allow delivery inside cells or subcellular compartments of said cells the nucleic acid molecules employed in accordance with the invention. These means include viral transduction, electroporation and also liposomal delivery means, polymeric carriers, chemical carriers, lipoplexes, polyplexes, dendrimers, nanoparticles, emulsion, natural endocytosis or phagocytose pathway as non-limiting examples.

In accordance with the present invention, the nucleic acid molecules detailed herein may be introduced in the T-cell by any suitable methods known in the art. Suitable, non-limiting methods for introducing a nucleic acid molecule into a T-cell according include stable transformation methods, wherein the nucleic acid molecule is integrated into the genome of the cell, transient transformation methods wherein the nucleic acid molecule is not integrated into the genome of the cell and virus mediated methods. Said nucleic acid molecule may be introduced into a cell by, for example, a recombinant viral vector (e.g., retroviruses, adenoviruses), liposome and the like. Transient transformation methods include, for example, microinjection, electroporation or particle bombardment. In certain embodiments, the nucleic acid molecule is a vector, such as a viral vector or plasmid. Suitably, said vector is an expression vector enabling the expression of the respective polypeptide(s) or protein(s) detailed herein by the T-cell.

A nucleic acid molecule introduced into the T-cell may be DNA or RNA. In certain embodiments, a nucleic acid molecule introduced into the T-cell is DNA. In certain embodiments, a nucleic acid molecule introduced into the T-cell is RNA, and in particular an mRNA encoding a polypeptide or protein detailed herein, which mRNA is introduced directly into the T-cell, for example by electroporation. A suitable electroporation technique is described, for example, in International Publication WO2013/176915 (in particular the section titled “Electroporation” bridging pages 29 to 30). A particular nucleic acid molecule which may be an mRNA is the nucleic acid molecule comprising a nucleotide sequence coding for a rare-cutting endonuclease able to selectively inactivate by DNA cleavage the gene encoding B2M. Another particular nucleic acid molecule which may be an mRNA is the nucleic acid molecule comprising a nucleotide sequence coding for a rare-cutting endonuclease able to selectively inactivate by DNA cleavage the gene encoding CIITA. A yet other particular nucleic acid molecule which may be an mRNA is the nucleic acid molecule comprising a nucleotide sequence coding for a rare-cutting endonuclease able to selectively inactivate by DNA cleavage at least one gene coding for one component of the T-Cell Receptor (TCR).

As a preferred embodiment of the invention, nucleic acid molecules encoding the endonucleases of the present invention are transfected under mRNA form in order to obtain transient expression and avoid chromosomal integration of foreign DNA, for example by electroporation. The inventors have determined different optimal conditions for mRNA electroporation in T-cell displayed in Table 1. The inventor used the cytoPulse technology which allows, by the use of pulsed electric fields, to transiently permeabilize living cells for delivery of material into the cells (U.S. Pat. No. 6,010,613 and WO 2004/083379). Pulse duration, intensity as well as the interval between pulses can be modified in order to reach the best conditions for high transfection efficiency with minimal mortality. Basically, the first high electric field pulses allow pore formation, while subsequent lower electric field pulses allow to moving the polynucleotide into the cell. In one aspect of the present invention, the inventor describe the steps that led to achievement of >95% transfection efficiency of mRNA in T cells, and the use of the electroporation protocol to transiently express different kind of proteins in T cells. In particular the invention relates to a method of transforming T cell comprising contacting said T cell with RNA and applying to T cell an agile pulse sequence consisting of:
(a) one electrical pulse with a voltage range from 2250 to 3000 V per centimeter, a pulse width of 0.1 ms and a pulse interval of 0.2 to 10 ms between the electrical pulses of step (a) and (b);
(b) one electrical pulse with a voltage range from 2250 to 3000 V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

In particular embodiment, the method of transforming T cell comprising contacting said T cell with RNA and applying to T cell an agile pulse sequence consisting of:
(a) one electrical pulse with a voltage of 2250, 2300, 2350, 2400, 2450, 2500, 2550, 2400, 2450, 2500, 2600, 2700, 2800, 2900 or 3000V per centimeter, a pulse width of 0.1 ms and a pulse interval of 0.2, 0.5, 1, 2, 3, 4, 5, 6, 7, 8, 9 or 10 ms between the electrical pulses of step (a) and (b);
(b) one electrical pulse with a voltage range from 2250, of 2250, 2300, 2350, 2400, 2450, 2500, 2550, 2400, 2450, 2500, 2600, 2700, 2800, 2900 or 3000V with a pulse width of 100 ms and a pulse interval of 100 ms between the electrical pulse of step (b) and the first electrical pulse of step (c); and
(c) 4 electrical pulses with a voltage of 325 V with a pulse width of 0.2 ms and a pulse interval of 2 ms between each of 4 electrical pulses.

Any values included in the value range described above are disclosed in the present application. Electroporation medium can be any suitable medium known in the art. Preferably, the electroporation medium has conductivity in a range spanning 0.01 to 1.0 milliSiemens.

TABLE 1
Different cytopulse programs used to determine the minimal voltage
required for electroporation in PBMC derived T-cells.

| | Group 1 | | | | Group 2 | | | | Group 3 | | | |
|---|---|---|---|---|---|---|---|---|---|---|---|---|
| Cytopulse | | | duration | Interval | | | duration | Interval | | | duration | Interval |
| program | Pulses | V | (ms) | (ms) | Pulses | V | (ms) | (ms) | Pulses | V | (ms) | (ms) |
| 1 | 1 | 600 | 0.1 | 0.2 | 1 | 600 | 0.1 | 100 | 4 | 130 | 0.2 | 2 |
| 2 | 1 | 900 | 0.1 | 0.2 | 1 | 900 | 0.1 | 100 | 4 | 130 | 0.2 | 2 |
| 3 | 1 | 1200 | 0.1 | 0.2 | 1 | 1200 | 0.1 | 100 | 4 | 130 | 0.2 | 2 |
| 4 | 1 | 1200 | 0.1 | 10 | 1 | 900 | 0.1 | 100 | 4 | 130 | 0.2 | 2 |
| 5 | 1 | 900 | 0.1 | 20 | 1 | 600 | 0.1 | 100 | 4 | 130 | 0.2 | 2 |

### Non Alloreactive T-Cells:

Although the method of the invention could be carried out in-vivo as part of a gene therapy, for instance, by using viral vectors targeting T-cells in blood circulation, which would include genetic sequences expressing a specific rare-cutting endonuclease along with other genetic sequences expressing, e.g., a CAR, the method of the invention is more generally intended to be practiced ex-vivo on cultured T-cells obtainable from patients or donors. The engineered T-cells engineered ex-vivo can be either re-implanted into a patient from where they originate, as part of an autologous treatment, or to be used as part of an allogeneic treatment. In this later case, it is preferable to further engineer the cells to make them non-alloreactive to ensure their proper engraftment. Accordingly, the method of the invention may include additional steps of procuring the T-cells from a donor and to inactivate genes thereof involved in MHC recognition and or being targets of immunosuppressive drugs such as described for instance in WO 2013/176915.

T-cell receptors (TCR) are cell surface receptors that participate in the activation of T-cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V (D) J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of GVHD. It has been shown that normal surface expression of the TCR depends on the coordinated synthesis and assembly of all seven components of the complex (Ashwell and Klusner 1990). The inactivation of TCR alpha or TCR beta can result in the elimination of the TCR from the surface of T cells preventing recognition of alloantigen and thus GVHD.

Thus, still according to the invention, engraftment of the T-cells may be improved by inactivating at least one gene encoding a TCR component. TCR is rendered not functional in the cells by inactivating TCR alpha gene and/or TCR beta gene(s).

With respect to the use of Cas9/CRISPR system, the inventors have determined appropriate target sequences within the 3 exons encoding TCR, allowing a significant reduction of toxicity in living cells, while retaining cleavage efficiency. The preferred target sequences are noted in Table 2 (+ for lower ratio of TCR negative cells, ++ for intermediate ratio, +++ for higher ratio).

TABLE 2
appropriate target sequences for the guide RNA using Cas9 in T-cells

| Exon | | | | | |
|---|---|---|---|---|---|
| TCR | Position | Strand | Target genomic sequence | SEQ ID | efficiency |
| Ex1 | 78 | −1 | GAGAATCAAAATCGGTGAATAGG | 8 | +++ |
| Ex3 | 26 | 1 | TTCAAAACCTGTCAGTGATTGGG | 9 | +++ |
| Ex1 | 153 | 1 | TGTGCTAGACATGAGGTCTATGG | 10 | +++ |
| Ex3 | 74 | −1 | CGTCATGAGCAGATTAAACCCGG | 11 | +++ |
| Ex1 | 4 | −1 | TCAGGGTTCTGGATATCTGTGGG | 12 | +++ |
| Ex1 | 5 | −1 | GTCAGGGTTCTGGATATCTGTGG | 13 | +++ |
| Ex3 | 33 | −1 | TTCGGAACCCAATCACTGACAGG | 14 | +++ |
| Ex3 | 60 | −1 | TAAACCCGGCCACTTTCAGGAGG | 15 | +++ |
| Ex1 | 200 | −1 | AAAGTCAGATTTGTTGCTCCAGG | 16 | ++ |
| Ex1 | 102 | 1 | AACAAATGTGTCACAAAGTAAGG | 17 | ++ |
| Ex1 | 39 | −1 | TGGATTTAGAGTCTCTCAGCTGG | 18 | ++ |
| Ex1 | 59 | −1 | TAGGCAGACAGACTTGTCACTGG | 19 | ++ |
| Ex1 | 22 | −1 | AGCTGGTACACGGCAGGGTCAGG | 20 | ++ |
| Ex1 | 21 | −1 | GCTGGTACACGGCAGGGTCAGGG | 21 | ++ |
| Ex1 | 28 | −1 | TCTCTCAGCTGGTACACGGCAGG | 22 | ++ |
| Ex3 | 25 | 1 | TTTCAAAACCTGTCAGTGATTGG | 23 | ++ |
| Ex3 | 63 | −1 | GATTAAACCCGGCCACTTTCAGG | 24 | ++ |
| Ex2 | 17 | −1 | CTCGACCAGCTTGACATCACAGG | 25 | ++ |
| Ex1 | 32 | −1 | AGAGTCTCTCAGCTGGTACACGG | 26 | ++ |
| Ex1 | 27 | −1 | CTCTCAGCTGGTACACGGCAGGG | 27 | ++ |
| Ex2 | 12 | 1 | AAGTTCCTGTGATGTCAAGCTGG | 28 | ++ |
| Ex3 | 55 | 1 | ATCCTCCTCCTGAAAGTGGCCGG | 29 | ++ |
| Ex3 | 86 | 1 | TGCTCATGACGCTGCGGCTGTGG | 30 | ++ |
| Ex1 | 146 | 1 | ACAAAACTGTGCTAGACATGAGG | 31 | + |
| Ex1 | 86 | −1 | ATTTGTTTGAGAATCAAAATCGG | 32 | + |
| Ex2 | 3 | −1 | CATCACAGGAACTTTCTAAAAGG | 33 | + |
| Ex2 | 34 | 1 | GTCGAGAAAAGCTTTGAAACAGG | 34 | + |
| Ex3 | 51 | −1 | CCACTTTCAGGAGGAGGATTCGG | 35 | + |
| Ex3 | 18 | −1 | CTGACAGGTTTTGAAAGTTTAGG | 36 | + |
| Ex2 | 43 | 1 | AGCTTTGAAACAGGTAAGACAGG | 37 | + |
| Ex1 | 236 | −1 | TGGAATAATGCTGTTGTTGAAGG | 38 | + |
| Ex1 | 182 | 1 | AGAGCAACAGTGCTGTGGCCTGG | 39 | + |
| Ex3 | 103 | 1 | CTGTGGTCCAGCTGAGGTGAGGG | 40 | + |
| Ex3 | 97 | 1 | CTGCGGCTGTGGTCCAGCTGAGG | 41 | + |
| Ex3 | 104 | 1 | TGTGGTCCAGCTGAGGTGAGGGG | 42 | + |
| Ex1 | 267 | 1 | CTTCTTCCCCAGCCCAGGTAAGG | 43 | + |
| Ex1 | 15 | −1 | ACACGGCAGGGTCAGGGTTCTGG | 44 | + |
| Ex1 | 177 | 1 | CTTCAAGAGCAACAGTGCTGTGG | 45 | + |
| Ex1 | 256 | −1 | CTGGGGAAGAAGGTGTCTTCTGG | 46 | + |
| Ex3 | 56 | 1 | TCCTCCTCCTGAAAGTGGCCGGG | 47 | + |
| Ex3 | 80 | 1 | TTAATCTGCTCATGACGCTGCGG | 48 | + |
| Ex3 | 57 | −1 | ACCCGGCCACTTTCAGGAGGAGG | 49 | + |
| Ex1 | 268 | 1 | TTCTTCCCCAGCCCAGGTAAGGG | 50 | + |
| Ex1 | 266 | −1 | CTTACCTGGGCTGGGGAAGAAGG | 51 | + |
| Ex1 | 262 | 1 | GACACCTTCTTCCCCAGCCCAGG | 52 | + |
| Ex3 | 102 | 1 | GCTGTGGTCCAGCTGAGGTGAGG | 53 | + |
| Ex3 | 51 | 1 | CCGAATCCTCCTCCTGAAAGTGG | 54 | + |

MHC antigens are also proteins that played a major role in transplantation reactions. Rejection is mediated by T cells reacting to the histocompatibility antigens on the surface of implanted tissues, and the largest group of these antigens is the major histocompatibility antigens (MHC). These proteins are expressed on the surface of all higher vertebrates and are called HLA antigens (for human leukocyte antigens) in human cells. Like TCR, the MHC proteins serve a vital role in T cell stimulation. Antigen presenting cells (often dendritic cells) display peptides that are the degradation products of foreign proteins on the cell surface on the MHC. In the presence of a co-stimulatory signal, the T cell becomes activated, and will act on a target cell that also displays that same peptide/MHC complex. For example, a stimulated T helper cell will target a macrophage displaying an antigen in conjunction with its MHC, or a cytotoxic T cell (CTL) will act on a virally infected cell displaying foreign viral peptides.

Thus, in order to provide less alloreactive T-cells, the method of the invention can further comprise the step of inactivating or mutating one HLA gene.

The class I HLA gene cluster in humans comprises three major loci, B, C and A, as well as several minor loci. The class II HLA cluster also comprises three major loci, DP, DQ and DR, and both the class I and class II gene clusters are polymorphic, in that there are several different alleles of both the class I and II genes within the population. There are also several accessory proteins that play a role in HLA functioning as well. The Tap1 and Tap2 subunits are parts of the TAP transporter complex that is essential in loading peptide antigens on to the class I HLA complexes, and the LMP2 and LMP7 proteosome subunits play roles in the proteolytic degradation of antigens into peptides for display on the HLA. Reduction in LMP7 has been shown to reduce the amount of MHC class I at the cell surface, perhaps through a lack of stabilization (Fehling et al. (1999) Science 265:1234-1237). In addition to TAP and LMP, there is the tapasin gene, whose product forms a bridge between the TAP complex and the HLA class I chains and enhances peptide loading. Reduction in tapasin results in cells with impaired MHC class I assembly, reduced cell surface expression of the MHC class I and impaired immune responses (Grandea et al. (2000) Immunity 13:213-222 and Garbi et al. (2000) Nat. Immunol. 1:234-238). Any of the above genes may be inactivated as part of the present invention as disclosed, for instance in WO 2012/012667.

Hence, in accordance with certain embodiments, the method of the invention further comprises inactivating at least one gene selected from the group consisting of RFXANK, RFX5, RFXAP, TAP1, TAP2, ZXDA, ZXDB and ZXDC. Inactivation may, for instance, be achieved by using a genome modification, more particularly through the expression in the T-cell of a rare-cutting endonuclease able to selectively inactivate by DNA cleavage a gene selected from the group consisting of RFXANK, RFX5, RFXAP, TAP1, TAP2, ZXDA, ZXDB and ZXDC.

### Activation and Expansion of T Cells

The method according to the invention may include a further step of activating and/or expanding the T-cell(s). This can be done prior to or after genetic modification of the T-cell(s), using the methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. According to these methods, the T cells of the invention can be expanded by contact with a surface having attached thereto an agent that stimulates a CD3 TCR complex associated signal and a ligand that stimulates a co-stimulatory molecule on the surface of the T cells.

In particular, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. As those of ordinary skill in the art can readily appreciate, the ratio of particles to cells may depend on particle size relative to the target cell. In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. Cell surface proteins may be ligated by allowing paramagnetic beads to which anti-CD3 and anti-CD28 are attached (3x28 beads) to contact the T cells. In one embodiment the cells (for example, 4 to 10 T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. The mixture may be cultured for several hours (about 3 hours) to about 14 days or any hourly integer value in between. In another embodiment, the mixture may be cultured for 21 days. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF,-10,-2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

### Therapeutic Applications

The T-cells obtainable in accordance with the present invention are intended to be used as a medicament, and in particular for treating, among others, cancer, infections (such viral infections) or immune diseases in a patient in need thereof. Accordingly, the present invention provides engineered T-cells for use as a medicament. Particularly, the present invention provides engineered T-cells for use in the treatment of a cancer, such as lymphoma, or viral infection. Also provided are compositions, particularly pharmaceutical compositions, which comprise at least one engineered T-cell of the present invention. In certain embodiments, a composition may comprise a population of engineered T-cell of the present invention.

The treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is particularly suited for allogenic immunotherapy, insofar as it enables the transformation of T-cells, typically obtained from donors, into non-alloreactive cells. This may be done under standard protocols and reproduced as many times as needed. The resulted modified T-cells may be pooled and administrated to one or several patients, being made available as an “off the shelf” therapeutic product.

The treatments are primarily to treat patients diagnosed with cancer. Cancers are preferably leukemias and lymphomas, which have liquid tumors, but may also concern solid tumors. Types of cancers to be treated with the genetically engineered T-cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

The treatment can take place in combination with one or more therapies selected from the group of antibodies therapy, chemotherapy, cytokines therapy, dendritic cell therapy, gene therapy, hormone therapy, laser light therapy and radiation therapy.

According to certain embodiments, T-cells of the invention can undergo robust in vivo T-cell expansion upon administration to a patient, and can persist in the body fluids for an extended amount of time, preferably for a week, more preferably for 2 weeks, even more preferably for at least one month. Although the T-cells according to the invention are expected to persist during these periods, their life span into the patient's body are intended not to exceed a year, preferably 6 months, more preferably 2 months, and even more preferably one month.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaliy, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In other embodiments, said effective amount of cells or composition comprising those cells are administrated parenterally. Said administration can be an intravenous administration. Said administration can be directly done by injection within a tumor.

In certain embodiments, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1 1; Henderson et al., Immun. 73:316-321, 1991; Bierer et al., Citrr. Opin. mm n. 5:763-773, 93). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded genetically engineered T-cells of the present invention. In an additional embodiment, expanded cells are administered before or following surgery.

Also encompassed within this aspect of the invention are methods for treating a patient in need thereof, comprising a) providing at least one engineered T-cell of the present invention, preferably a population of said T-cell; and b) administering said T-cell or population to said patient.

Also encompassed within this aspect of the invention are methods for preparing a medicament using at least one engineered T-cell of the present invention, and preferably a population of said T-cell. Accordingly, the present invention provides the use of at least one engineered T-cell of the present invention, and preferably a population of said T-cell, in the manufacture of a medicament. Preferably, such medicament is for use in the treatment of a cancer, such as lymphoma, or viral infection.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
by “polynucleotide successively comprising a first region of homology to sequences upstream of said double-stranded break, a sequence to be inserted in the genome of said cell and a second region of homology to sequences downstream of said double-stranded break” it is intended to mean a DNA construct or a matrix comprising a first and second portion that are homologous to regions 5′ and 3′ of a DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the targeted gene comprised in the locus of interest and this matrix, wherein the genomic sequence containing the DNA target is replaced by the third portion of the matrix and a variable part of the first and second portions of said matrix.
by “DNA target”, “DNA target sequence”, “target DNA sequence”, “nucleic acid target sequence”, “target sequence”, or “processing site” is intended a polynucleotide sequence that can be targeted and processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, or penetrating peptides. In these later cases, delivery vectors are molecule carriers.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non limiting example, that are able to integrate the genome of a target cell. At the opposite by “non integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
By “cell” or “cells” is intended any eukaryotic living cells, primary cells and cell lines derived from these organisms for in vitro cultures.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.

As non-limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; Hela cells; HT-1080 cells; HCT-116 cells; Hu-h7 cells; Huvec cells; Molt 4 cells.

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.
by “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.
As used herein, the term “locus” is the specific physical location of a DNA sequence (e.g. of a gene) on a chromosome. The term “locus” can refer to the specific physical location of a rare-cutting endonuclease target sequence on a chromosome. Such a locus can comprise a target sequence that is recognized and/or cleaved by a rare-cutting endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.
The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
By “fusion protein” is intended the result of a well-known process in the art consisting in the joining of two or more genes which originally encode for separate proteins or part of them, the translation of said “fusion gene” resulting in a single polypeptide with functional properties derived from each of the original proteins.
“identity” refers to sequence identity between two nucleic acid molecules or polypeptides. Identity can be determined by comparing a position in each sequence which may be aligned for purposes of comparison. When a position in the compared sequence is occupied by the same base or amino acid, then the molecules are identical at that position. A degree of similarity or identity between nucleic acid or amino acid sequences is a function of the number of identical or matching nucleotides or amino acids at positions shared by the nucleic acid or amino acid sequences, respectively. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
“inhibiting” or “inhibit” expression of B2M means that the expression of B2M in the cell is reduced by at least 1%, at least 5%, at least 10%, at least 20%, at least 30%, at least 40%, at least 50% at least 60%, at least 70%, at least 80%, at least 90%, at least 95%, at least 99% or 100%. More particularly, “inhibiting” or “inhibit” expression of B2M means that the amount of B2M in the cell is reduced by at least 1%, at least 5%, at least 10%, at least 20%, at least 30%, at least 40%, at least 50% at least 60%, at least 70%, at least 80%, at least 90%, at least 95%, at least 99% or 100%. The expression or amount of protein in a cell can be determined by any suitable means know in the art, such as ELISA, Immunohistochemistry, Western Blotting or Flow Cytometry using B2M specific antibodies. Such antibodies are commercially available from various sources, such from Merck Millipore, Billerica, MA, USA; or Abcam plc, Cambridge, UK.
“inhibiting” or “inhibit” expression of CIITA means that the expression of CIITA in the cell is reduced by at least 1%, at least 5%, at least 10%, at least 20%, at least 30%, at least 40%, at least 50% at least 60%, at least 70%, at least 80%, at least 90%, at least 95%, at least 99% or 100%. More particularly, “inhibiting” or “inhibit” expression of CIITA means that the amount of CIITA in the cell is reduced by at least 1%, at least 5%, at least 10%, at least 20%, at least 30%, at least 40%, at least 50% at least 60%, at least 70%, at least 80%, at least 90%, at least 95%, at least 99% or 100%. The expression or amount of protein in a cell can be determined by any suitable means know in the art, such as ELISA, Immunohistochemistry, Western Blotting or Flow Cytometry using CIITA specific antibodies. Such antibodies are commercially available from various sources, such from Abcam plc, Cambridge, UK; or Santa Cruz Biotechnology, Inc., Santa Cruz, CA, USA.
“signal-transducing domain” or “co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory igand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, a ligand that specifically binds with CD83.
A “co-stimulatory molecule” refers to the cognate binding partner on a Tcell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.
A “co-stimulatory signal” as used herein refers to a signal, which in combination with primary signal, such as TCR/CD3 ligation, leads to T cell proliferation and/or upregulation or downregulation of key molecules.
“bispecific antibody” refers to an antibody that has binding sites for two different antigens within a single antibody molecule. It will be appreciated by those skilled in the art that other molecules in addition to the canonical antibody structure may be constructed with two binding specificities. It will further be appreciated that antigen binding by bispecific antibodies may be simultaneous or sequential. Bispecific antibodies can be produced by chemical techniques (see e.g., Kranz et al. (1981) Proc. Natl. Acad. Sci. USA 78, 5807), by “polydoma” techniques (See U.S. Pat. No. 4,474,893) or by recombinant DNA techniques, which all are known per se. As a non-limiting example, each binding domain comprises at least one variable region from an antibody heavy chain (“VH or H region”), wherein the VH region of the first binding domain specifically binds to the lymphocyte marker such as CD3, and the VH region of the second binding domain specifically binds to tumor antigen.
The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells.
The term “subject” or “patient” as used herein includes all members of the animal kingdom including non-human primates and humans.
The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### Examples

### Tale-Nucleases Cleaving Human CIITA

mRNA encoding the TALE-nucleases targeting exons of the human CIITA gene were ordered from Cellectis Bioresearch (8, rue de la Croix Jarry, 75013 PARIS). Table 3 below indicates the target sequences cleaved by each of the two independent entities (called half TALE-nucleases) each containing a repeat sequence engineered to bind and cleave between target sequences consisting of two 17-bp long sequences (called half targets) separated by a 15-bp spacer. Because Exon 2 and 3 are shared by all transcript variants of CIITA, two TALEN pairs were designed for Exon 2 and 3. No obvious offsite targeting in the human genome have been predicted using TALE-Nucleases targeting these sequences.

TABLE 3
Description of the CIITA TALE-nucleases
and related target sequences

| Target name | Target sequence |
|---|---|
| TALEN 1_Exon 2_CMH-II-TA | TTCCCTCCCAGGCAGCTC |
| | acagtgtgccaccaTGGA |
| | GTTGGGGCCCCTA |
| | (SEQ ID NO: 55) |
| TALEN 2_Exon 2_CMH-II-TA | TGCCTCTACCACTTCTAT |
| | gaccagatggacctGGCT |
| | GGAGAAGAAGAGA |
| | (SEQ ID NO: 56) |
| TALEN 1_Exon3_CMH-II-TA | 5′TCTTCATCCAAGGGAC |
| | TTttcctcccagaaccCG |
| | ACACAGACACCATCA |
| | (SEQ ID NO: 57) |
| TALEN 2_Exon3_CMH-II-TA | TGTTGTGTGACATGGAAG |
| | gtgatgaagagaccAGGG |
| | AGGCTTATGCCAA |
| | (SEQ ID NO: 58) |

### Tale-Nucleases Cleaving Human B2m

mRNA encoding the TALE-nucleases targeting exons of the human β2m gene were ordered from Cellectis Bioresearch (8, rue de la Croix Jarry, 75013 PARIS). Table 4 below indicates the target sequences cleaved by each of the two independent entities (called half TALE-nucleases) each containing a repeat sequence engineered to bind and cleave between target sequences consisting of two 17-bp long sequences (called half targets) separated by a 15-bp spacer.

TABLE 4
Description of the β2m TALE-nucleases
and related target sequences

| Target | | Half TALE-nuclease |
|---|---|---|
| name | Target sequence | sequence |
| B2M_T03 | 5′-CCAAAGATTCAGGTTT | Repeat B2M_T03-L |
| | actcacgtcatccagc | (pCLS24605) |
| | (spacer) | SEQ ID NO: 67 |
| | AGAGAATGGAAAGTC-3′ | B2M_T03-R |
| | (SEQ ID NO: 59) | (pCLS24606) |
| | | SEQ ID NO: 68 |

### Tale-Nucleases Cleaving Human TCR Genes (TRAC and TRBC)

The human genome contains two functional T-cell receptor beta chains (TRBC1 and TRBC2). During the development of alpha/beta T lymphocytes, one of these two constant chains is selected in each cell to be spliced to the variable region of TCR-beta and form a functional full length beta chain. Table 5 below presents a TRAC and 2 TRBC target sequences and their corresponding TALEN sequences. The 2 TRBC targets were chosen in sequences conserved between TRBC1 and TRBC2 so that the corresponding TALE-nuclease would cleave both TRBC1 and TRBC2 at the same time.

TABLE 5
Description of the TRAC and TRBC TALE-nucleases
and sequences of the TALE-nucleases
target sites in the human corresponding genes.

| Target | Target sequence | Half TALE-nuclease |
|---|---|---|
| TRAC_T01 | TTGTCCCACAGATATCC | TRAC_T01-L TALEN |
| | Agaaccctgaccctg | (SEQ ID NO: 69) |
| | CCGTGTACCAGCTGAGA | TRAC_T01-R TALEN |
| | (SEQ ID NO: 60) | (SEQ ID NO: 70) |
| TRBC_T01 | TGTGTTTGAGCCATCAG | TRBC_T01-L TALEN |
| | aagcagagatctccc | (SEQ ID NO: 71) |
| | ACACCCAAAAGGCCACA | TRBC_T01-R TALEN |
| | (SEQ ID NO: 61) | (SEQ ID NO: 72) |
| TRBC_T02 | TTCCCACCCGAGGTCGC | TRBC_T02-L TALEN |
| | tgtgtttgagccatca | (SEQ ID NO: 73) |
| | GAAGCAGAGATCTCCCA | TRBC_T02-R TALEN |
| | (SEQ ID NO: 62) | (SEQ ID NO: 74) |

Other target sequences in TRAC and CD52 genes have been designed, which are displayed in Table 6.

TABLE 6
Additional target sequences
for TRAC TALE-nucleases.

| Target | Target sequence |
|---|---|
| TRAC_T02 | TTTAGAAAGTTCCTGTG |
| | atgtcaagctggtcg |
| | AGAAAAGCTTTGAAACA |
| | (SEQ ID NO: 63) |
| TRAC_T03 | TCCAGTGACAAGTCTGT |
| | ctgcctattcaccga |
| | TTTTGATTCTCAAACAA |
| | (SEQ ID NO: 64) |
| TRAC_T04 | TATATCACAGACAAAAC |
| | tgtgctagacatgag |
| | GTCTATGGACTTCAAGA |
| | (SEQ ID NO: 65) |
| TRAC_T05 | TGAGGTCTATGGACTTC |
| | aagagcaacagtgct |
| | GTGGCCTGGAGCAACAA |
| | (SEQ ID NO: 66) |

Electroporation of mRNA of purified Tcells activated using Cytopulse Technology

After determining the best cytopulse program that allows an efficient DNA electroporation of T cells, we tested whether this method was applicable to the mRNA electroporation.

5×106 purified T cells preactivated 6 days with PHA/IL2 were resupended in cytoporation buffer T (BTX-Harvard apparatus) and electroporated in 0.4 cm cuvettes with 10 ug of mRNA encoding GFP or 20 μg of plasmids encoding GFP or pUC using the preferred cytopulse program of table 7.

TABLE 7
Cytopulse program used to electroporate purified T-cells.

| | Group 1 | | | | Group 2 | | | | Group 3 | | | |
|---|---|---|---|---|---|---|---|---|---|---|---|---|
| Cytopulse | | | duration | Interval | | | duration | Interval | | | duration | Interval |
| program | Pulse | V | (ms) | (ms) | Pulse | V | (ms) | (ms) | Pulse | V | (ms) | (ms) |
| 3 | 1 | 1200 | 0.1 | 0.2 | 1 | 1200 | 0.1 | 100 | 4 | 130 | 0.2 | 2 |

48h after transfection cells were stained with viability dye (eFluor-450) and the cellular viability and % of viable GFP+ cells was determined by flow cytometry.

The electroporation of RNA with the optimal condition determined here was not toxic and allowed transfection of more than 95% of the viable cells.

In synthesis, the whole dataset shows that T-cells can be efficiently transfected either with DNA or RNA. In particular, RNA transfection has no impact on cellular viability and allows uniform expression levels of the transfected gene of interest in the cellular population.

Efficient transfection can be achieved early after cellular activation, independently of the activation method used (PHA/IL-2 or CD3/CD28-coated-beads). The inventors have succeeded in transfecting cells from 72h after activation with efficiencies of >95%. In addition, efficient transfection of T cells after thawing and activation can also be obtained using the same electroporation protocol.

mRNA Electroporation in Primary Human T Cells for TALE-Nuclease Functional Expression

After demonstrating that mRNA electroporation allow efficient expression of GFP in primary human T cells, we tested whether this method was applicable to the expression of other proteins of interest. Transcription activator-like effector nucleases (TALE-nuclease) are site-specific nucleases generated by the fusion of a TAL DNA binding domain to a DNA cleavage domain. They are powerful genome editing tools as they induce double-strand breaks at practically any desired DNA sequence. These double-strand breaks activate Non-homologous end-joining (NHEJ), an error-prone DNA repair mechanism, potentially leading to inactivation of any desired gene of interest. Alternatively, if an adequate repair template is introduced into the cells at the same time, TALE-nuclease-induced DNA breaks can be repaired by homologous recombination, therefore offering the possibility of modifying at will the gene sequence.

We have used mRNA electroporation to express a TALE-nuclease designed to specifically cleave a sequence in the human gene coding for the alpha chain of the T cell antigen receptor (TRAC). Mutations induced in this sequence are expected to result in gene inactivation and loss of TCRαβ complex from the cell surface. TRAC TALE-nuclease RNA or non-coding RNA as control are transfected into activated primary human T lymphocytes using Cytopulse technology. The electroporation sequence consisted in 2 pulses of 1200 V followed by four pulses of 130 V as described in Table 7.

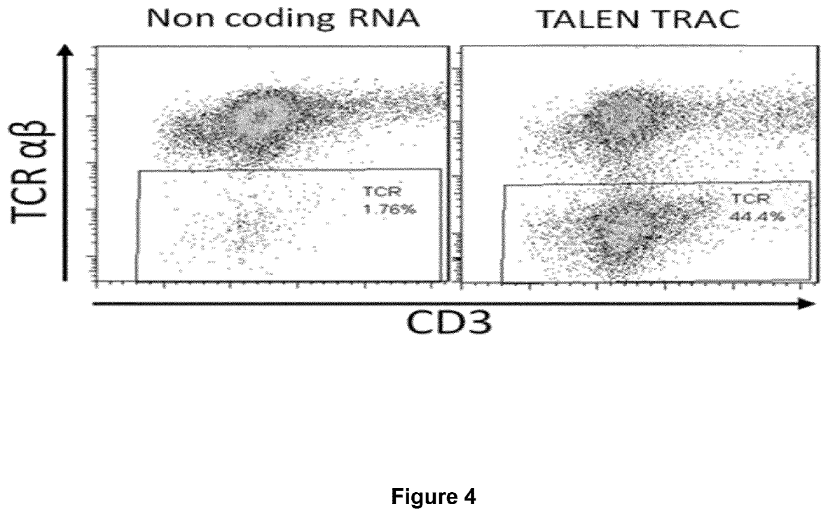

By flow cytometry analysis of TCR surface expression 7 days post electroporation (FIG. 4, top panel), we observed that 44% of T cells lost the expression of TCRαβ. We analyzed the genomic DNA of the transfected cells by PCR amplification of the TRAC locus followed by 454 high throughput sequencing. 33% of alleles sequenced (727 out of 2153) contained insertion or deletion at the site of TALE-nuclease cleavage.

These data indicate that electroporation of mRNA using cytopulse technology results in functional expression of TRAC TALE-nuclease.

### Activity of TRAC-TALE-Nuclease and TRBC-TALE-Nuclease in HEK293 Cells

Each TALE-nuclease construct was subcloned using restriction enzyme digestion in a mammalian expression vector under the control of pEF1alpha long promoter. One million HEK293 cells were seeded one day prior to transfection. Cells were transfected with 2.5 μg of each of the two plasmids encoding the TALE-nucleases recognizing the two half targets in the genomic sequence of interest in the T-cell receptor alpha constant chain region (TRAC) or T-cell receptor beta constant chain region (TRBC) under the control of the EF1-alpha promoter or 5 μg of a control pUC vector (pCLS0003) using 25 μl of lipofectamine (Invitrogen) according to the manufacturer's instructions.

The double stranded cleavage generated by TALE-nucleases in TRAC coding sequences is repaired in live cells by non homologous end joining (NHEJ), which is an error-prone mechanism. Activity of TALE-nucleases in live cells is measured by the frequency of insertions or deletions at the genomic locus targeted. 48 hours after transfection, genomic DNA was isolated from transfected cells and locus specific PCRs were performed using the following primers: for TRAC: 5′-ATCACTGGCATCTGGACTCCA-3′ (SEQ ID NO: 75), for TRBC1: 5′-AGAGCCCCTACCAGAACCAGAC-3′ (SEQ ID NO: 76, or for TRBC2: 5′-GGACCTAGTAACATAATTGTGC-3′ (SEQ ID NO: 77), and the reverse primer for TRAC: 5′-CCTCATGTCTAGCACAGTTT-3′ (SEQ ID NO: 78), for TRBC1 and TRBC2: 5′-ACCAGCTCAGCTCCACGTGGT-3′ (SEQ ID NO: 79). PCR products were sequenced by a 454 sequencing system (454 Life Sciences). Approximately 10,000 sequences were obtained per PCR product and then analyzed for the presence of site-specific insertion or deletion events; results are in Table 8.

TABLE 8
Percentages of indels for TALE-nuclease targeting
TRAC_T01, TRBC_T01 and TRBC_T02 targets.

| | % Indels with | % Indels with |
|---|---|---|
| | TALE-nuclease | pUC control |
| Target | transfection | transfection |
| TRAC_T01 | 41.9 | 0.3 |
| TRBC_T01 in constant chain 1 | 3.81 | 0 |
| TRBC_T01 in constant chain 2 | 2.59 | 0 |
| TRBC_T02 in constant chain 1 | 14.7 | 0 |
| TRBC_T02 in constant chain 1 | 5.99 | 0 |

### Activity of β2m and TRAC-TALE-Nuclease in Primary T Lymphocytes

Each TALE-nuclease construct was subcloned using restriction enzyme digestion in a mammalian expression vector under the control of the T7 promoter.

mRNA encoding TALE-nuclease cleaving β2m, TRAC and TRBC genomic sequence were synthesized from plasmid carrying the coding sequences downstream from the T7 promoter. T lymphocytes isolated from peripheral blood were activated for 5 days using anti-CD3/CD28 activator beads (Life technologies) and 5 million cells were then transfected by electroporation with 10 μg of each of 2 mRNAs encoding both half TALE-nuclease (or non coding RNA as controls) using a CytoLVT-P instrument. As a consequence of the insertions and deletions induced by NHEJ, the coding sequence for β2m and/or TRAC will be out of frame in a fraction of the cells resulting in non-functional genes. 5 days after electroporation, cells were labeled with fluorochrome-conjugated anti-β2m or anti-TCR antibody by flow cytometry for the presence of β2m or TCR at their cell surface. Since all T lymphocytes expanded from peripheral blood normally express β2m and TCR, the proportion of β2m-negative or TCR-negative cells is a direct measure of TALE-nuclease activity.

Functional Analysis of T Cells with Targeted TRAC Gene

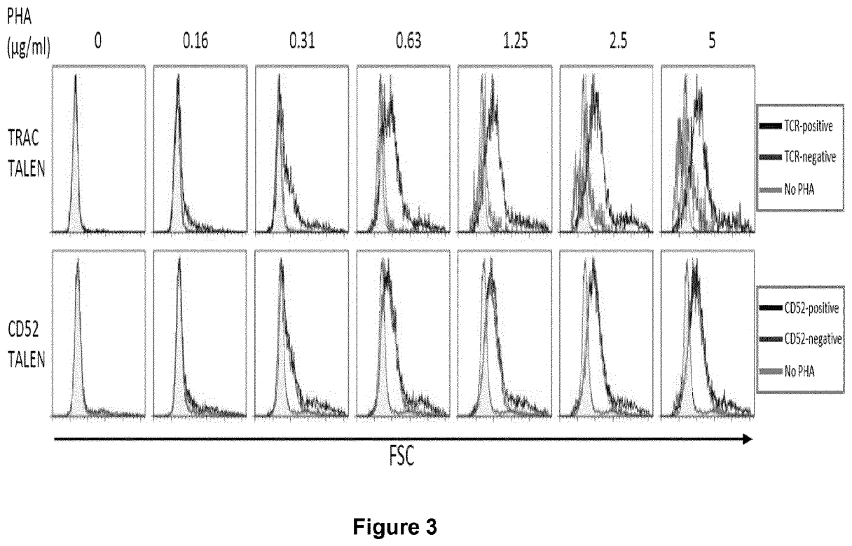

The goal of TRAC gene inactivation is to render T lymphocytes unresponsive to T-cell receptor stimulation. As described in the previous paragraph, T lymphocytes were transfected with mRNA encoding TALE-nuclease cleaving TRAC. 16 days after transfection, cells were treated with up to 5 μg/ml of phytohemagglutinin (PHA, Sigma-Aldrich), a T-cell mitogen acting through the T cell receptor. Cells with a functional T-cell receptor should increase in size following PHA treatment. After three days of incubation, cells were labeled with a fluorochrome-conjugated anti-TCR antibody and analyzed by flow cytometry to compare the cell size distribution between TCR-positive and TCR-negative cells. FIG. 3 shows that TCR-positive cells significantly increase in size after PHA treatment whereas TCR-negative cells have the same size as untreated cells indicating that TRAC inactivation rendered them unresponsive to TCR-signaling.

Functional Analysis of T Cells with Targeted β2m Gene

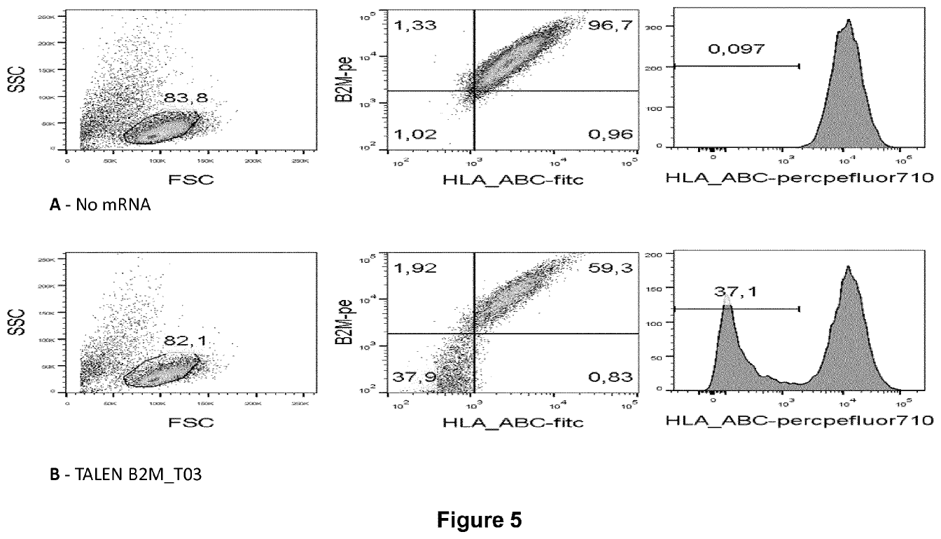

Similarly to the above, the TALEN-transfected cells and control cells (transfected without RNA) were stained with fluorochrome labeled antibody against β2M protein as well as an antibody recognizing all three classes MHC-I molecules (HLA-A, -B or -C). TALEN transfection induced loss of surface expression of β2M and MHC-I molecules in more than 37% of T cells. See FIG. 5

Genomic Safety of β2m-TALE-Nuclease and TRAC-TALE-Nuclease in Primary T Lymphocytes

As our constructs include nuclease subunits, an important question is whether multiple TALE-nuclease transfection can lead to genotoxicity and off-target cleavage at ‘close match’ target sequences or by mispairing of half-TALE-nucleases. To estimate the impact of TRAC-TALE-nuclease and β2m-TALE-nuclease on the integrity of the cellular genomes, we listed sequences in the human genome that presented the potential for off-site cleavage. To generate this list, we identified all the sequences in the genome with up to 4 substitutions compared to the original half targets and then identified the pairs of potential half targets in a head to head orientation with a spacer of 9 to 30 bp from each other. This analysis included sites potentially targeted by homodimers of one half-TALE-nuclease molecule or heterodimers formed by one β2m half TALE-nuclease and one TRAC half-TALE-nuclease. We scored the potential off-site targets based on the specificity data taking into account the cost of individual substitutions and the position of the substitutions (where mismatches are better tolerated for bases at the 3′ end of the half target). We obtained 173 unique sequences with a score reflecting an estimation of the likelihood of cleavage. We selected the 15 top scores and analyzed by deep sequencing the frequency of mutations found at these loci in T cells simultaneously transfected with β2m and TRAC TALE-nuclease and purified by magnetic separation as β2m-negative, TCRαβ-negative. Results showed that the highest frequency of insertion/deletion is 7×10-4. These results make the putative offsite target at least 600 times less likely to be mutated than the intended targets. The TALE-nuclease reagents used in this study therefore appear extremely specific.

Electroporation of T Cells with a Monocistronic mRNA Encoding for an Anti-CD19 Single Chain Chimeric Antigen Receptor (CAR):

5×106 T cells preactivated several days (3-5) with anti-CD3/CD28 coated beads and IL2 were resuspended in cytoporation buffer T, and electroporated in 0.4 cm cuvettes without mRNA or with 10 μg of mRNA encoding a single chain CAR (SEQ ID NO: 6) using the program described in Table 7.

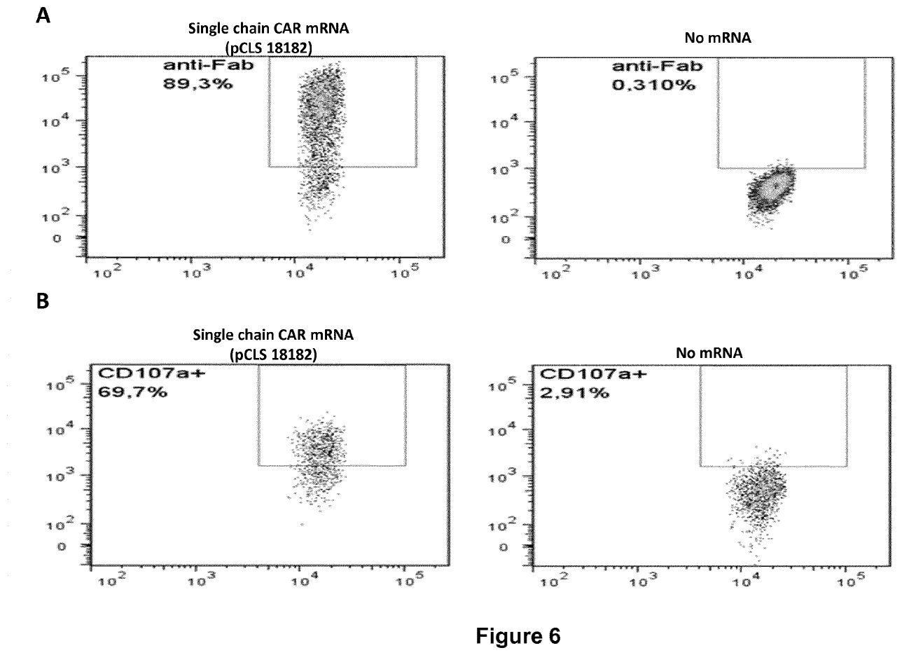

24 hours post electroporation, cells were stained with a fixable viability dye eFluor-780 and a PE-conjugated goat anti mouse IgG F(ab′)2 fragment specific to assess the cell surface expression of the CAR on the live cells. The data is shown in the FIG. 6. A indicates that the vast majority of the live T cells electroporated with the monocitronic mRNA described previously express the CAR at their surface. 24 hours post electroporation, T cells were cocultured with Daudi (CD19+) cells for 6 hours and analyzed by flow cytometry to detect the expression of the degranulation marker CD107a at their surface.

The data shown in FIG. 6 indicates that the majority of the cells electroporated with the monocistronic mRNA described previously degranulate in the presence of target cells expressing CD19. These results clearly demonstrate that the CAR expressed at the surface of electroporated T cells is active.

In the following examples, to prolong their survival and enhance their therapeutic activity, the inventors describe a method to prevent NK-cell mediated rejection of therapeutic allogeneic T cells by engineering the allogenic T cells through the inactivation of the β2M gene using specific TALEN, combined to either: i) the expression of a chimeric single chain molecule composed of UL18 and β2M β2M-UL18) or ii) the secretion of NKG2D ligands. The particularity resides in applying to primary T cells a mechanism occurring normally in tumor cells or virally infected cells. Thus, the mechanism of action is potentially different: in tumor cells, shedding NKG2D ligands leads to their decreased presence at the surface whereas in engineered cells, secreted the NKG2D ligand(s) would serve as a decoy for several other NKG2D ligands potentially still present at the T cell surface . . .

### Efficient β2M Gene Knock Out Using Specific β2M TALEN.

Specific TALEN targeting a sequence (T01, SEQ ID N°81) within the first coding exon of the β2M gene (GenBank accession number NC_000015) has been produced (left DNA binding domain RVDs: NN—NN-HD-HD-NG-NG-NI—NN-HD-NG-NN-NG-NN-HD-NG-NG with SEQ ID NO: 82, and right DNA binding domain RVDs: NI—NN-HD-HD-NG-HD-HD-NI—NN-NN-HD-HD-NI—NN-NI-NG with SEQ ID NO: 83). The Table 9 below reports sequences for T01 targeting sequence, as well as for 2 additional targets T02 and T03 and their corresponding left and right TALE sequences.

TABLE 9
Description of additional β2m TALE-nucleases sequences

| Target | SEQ | |
|---|---|---|
| name | ID NO: | Half TALE-nuclease sequence |
| T01 | 80 | TCTCGCTCCGTGGCCTTAGCTGTGCTCGCGCTACTCTCTCTTTCTGGCCTGGAGGCTA |
| Beta2M | | |
| target | | |
| T01 | 81 | ATGGGCGATCCTAAAAAGAAACGTAAGGTCATCGATTACCCATACGATGTTCCAGATTACGCTATCGATA |
| TALEN | | TCGCCGATCTACGCACGCTCGGCTACAGCCAGCAGCAACAGGAGAAGATCAAACCGAAGGTTCGTTCGA |
| Beta2M | | CAGTGGCGCAGCACCACGAGGCACTGGTCGGCCACGGGTTTACACACGCGCACATCGTTGCGTTAAGCC |
| LEFT | | AACACCCGGCAGCGTTAGGGACCGTCGCTGTCAAGTATCAGGACATGATCGCAGCGTTGCCAGAGGCGA |
| | | CACACGAAGCGATCGTTGGCGTCGGCAAACAGTGGTCCGGCGCACGCGCTCTGGAGGCCTTGCTCACGG |
| | | TGGCGGGAGAGTTGAGAGGTCCACCGTTACAGTTGGACACAGGCCAACTTCTCAAGATTGCAAAACGTG |
| | | GCGGCGTGACCGCAGTGGAGGCAGTGCATGCATGGCGCAATGCACTGACGGGTGCCCCGCTCAACTTGA |
| | | CCCCCCAGCAGGTGGTGGCCATCGCCAGCAATAATGGTGGCAAGCAGGCGCTGGAGACGGTCCAGCGG |
| | | CTGTTGCCGGTGCTGTGCCAGGCCCACGGCTTGACCCCCCAGCAGGTGGTGGCCATCGCCAGCAATAATG |
| | | GTGGCAAGCAGGCGCTGGAGACGGTCCAGCGGCTGTTGCCGGTGCTGTGCCAGGCCCACGGCTTGACCC |
| | | CGGAGCAGGTGGTGGCCATCGCCAGCCACGATGGCGGCAAGCAGGCGCTGGAGACGGTCCAGCGGCTG |
| | | TTGCCGGTGCTGTGCCAGGCCCACGGCTTGACCCCGGAGCAGGTGGTGGCCATCGCCAGCCACGATGGC |
| | | GGCAAGCAGGCGCTGGAGACGGTCCAGCGGCTGTTGCCGGTGCTGTGCCAGGCCCACGGCTTGACCCCC |
| | | CAGCAGGTGGTGGCCATCGCCAGCAATGGCGGTGGCAAGCAGGCGCTGGAGACGGTCCAGCGGCTGTT |
| | | GCCGGTGCTGTGCCAGGCCCACGGCTTGACCCCCCAGCAGGTGGTGGCCATCGCCAGCAATGGCGGTGG |
| | | CAAGCAGGCGCTGGAGACGGTCCAGCGGCTGTTGCCGGTGCTGTGCCAGGCCCACGGCTTGACCCCGGA |
| | | GCAGGTGGTGGCCATCGCCAGCAATATTGGTGGCAAGCAGGCGCTGGAGACGGTGCAGGCGCTGTTGC |
| | | CGGTGCTGTGCCAGGCCCACGGCTTGACCCCCCAGCAGGTGGTGGCCATCGCCAGCAATAATGGTGGCA |
| | | AGCAGGCGCTGGAGACGGTCCAGCGGCTGTTGCCGGTGCTGTGCCAGGCCCACGGCTTGACCCCGGAGC |
| | | AGGTGGTGGCCATCGCCAGCCACGATGGCGGCAAGCAGGCGCTGGAGACGGTCCAGCGGCTGTTGCCG |
| | | GTGCTGTGCCAGGCCCACGGCTTGACCCCCCAGCAGGTGGTGGCCATCGCCAGCAATGGCGGTGGCAAG |
| | | CAGGCGCTGGAGACGGTCCAGCGGCTGTTGCCGGTGCTGTGCCAGGCCCACGGCTTGACCCCCCAGCAG |
| | | GTGGTGGCCATCGCCAGCAATAATGGTGGCAAGCAGGCGCTGGAGACGGTCCAGCGGCTGTTGCCGGT |
| | | GCTGTGCCAGGCCCACGGCTTGACCCCCCAGCAGGTGGTGGCCATCGCCAGCAATGGCGGTGGCAAGCA |
| | | GGCGCTGGAGACGGTCCAGCGGCTGTTGCCGGTGCTGTGCCAGGCCCACGGCTTGACCCCCCAGCAGGT |
| | | GGTGGCCATCGCCAGCAATAATGGTGGCAAGCAGGCGCTGGAGACGGTCCAGCGGCTGTTGCCGGTGCT |
| | | GTGCCAGGCCCACGGCTTGACCCCGGAGCAGGTGGTGGCCATCGCCAGCCACGATGGCGGCAAGCAGG |
| | | CGCTGGAGACGGTCCAGCGGCTGTTGCCGGTGCTGTGCCAGGCCCACGGCTTGACCCCCCAGCAGGTGG |
| | | TGGCCATCGCCAGCAATGGCGGTGGCAAGCAGGCGCTGGAGACGGTCCAGCGGCTGTTGCCGGTGCTGT |
| | | GCCAGGCCCACGGCTTGACCCCTCAGCAGGTGGTGGCCATCGCCAGCAATGGCGGCGGCAGGCCGGCGC |
| | | TGGAGAGCATTGTTGCCCAGTTATCTCGCCCTGATCCGGCGTTGGCCGCGTTGACCAACGACCACCTCGTC |
| | | GCCTTGGCCTGCCTCGGCGGGCGTCCTGCGCTGGATGCAGTGAAAAAGGGATTGGGGGATCCTATCAGC |
| | | CGTTCCCAGCTGGTGAAGTCCGAGCTGGAGGAGAAGAAATCCGAGTTGAGGCACAAGCTGAAGTACGTG |
| | | CCCCACGAGTACATCGAGCTGATCGAGATCGCCCGGAACAGCACCCAGGACCGTATCCTGGAGATGAAG |
| | | GTGATGGAGTTCTTCATGAAGGTGTACGGCTACAGGGGCAAGCACCTGGGCGGCTCCAGGAAGCCCGAC |
| | | GGCGCCATCTACACCGTGGGCTCCCCCATCGACTACGGCGTGATCGTGGACACCAAGGCCTACTCCGGCG |
| | | GCTACAACCTGCCCATCGGCCAGGCCGACGAAATGCAGAGGTACGTGGAGGAGAACCAGACCAGGAAC |
| | | AAGCACATCAACCCCAACGAGTGGTGGAAGGTGTACCCCTCCAGCGTGACCGAGTTCAAGTTCCTGTTCG |
| | | TGTCCGGCCACTTCAAGGGCAACTACAAGGCCCAGCTGACCAGGCTGAACCACATCACCAACTGCAACGG |
| | | CGCCGTGCTGTCCGTGGAGGAGCTCCTGATCGGCGGCGAGATGATCAAGGCCGGCACCCTGACCCTGGA |
| | | GGAGGTGAGGAGGAAGTTCAACAACGGCGAGATCAACTTCGCGGCCGACTGATAA |
| T01 | 82 | ATGGGCGATCCTAAAAAGAAACGTAAGGTCATCGATAAGGAGACCGCCGCTGCCAAGTTCGAGAGACAG |
| TALEN | | CACATGGACAGCATCGATATCGCCGATCTACGCACGCTCGGCTACAGCCAGCAGCAACAGGAGAAGATC |
| Beta2M | | AAACCGAAGGTTCGTTCGACAGTGGCGCAGCACCACGAGGCACTGGTCGGCCACGGGTTTACACACGCG |
| RIGHT | | CACATCGTTGCGTTAAGCCAACACCCGGCAGCGTTAGGGACCGTCGCTGTCAAGTATCAGGACATGATCG |
| | | CAGCGTTGCCAGAGGCGACACACGAAGCGATCGTTGGCGTCGGCAAACAGTGGTCCGGCGCACGCGCTC |
| | | TGGAGGCCTTGCTCACGGTGGCGGGAGAGTTGAGAGGTCCACCGTTACAGTTGGACACAGGCCAACTTC |
| | | TCAAGATTGCAAAACGTGGCGGCGTGACCGCAGTGGAGGCAGTGCATGCATGGCGCAATGCACTGACG |
| | | GGTGCCCCGCTCAACTTGACCCCGGAGCAGGTGGTGGCCATCGCCAGCAATATTGGTGGCAAGCAGGCG |
| | | CTGGAGACGGTGCAGGCGCTGTTGCCGGTGCTGTGCCAGGCCCACGGCTTGACCCCCCAGCAGGTGGTG |
| | | GCCATCGCCAGCAATAATGGTGGCAAGCAGGCGCTGGAGACGGTCCAGCGGCTGTTGCCGGTGCTGTGC |
| | | CAGGCCCACGGCTTGACCCCGGAGCAGGTGGTGGCCATCGCCAGCCACGATGGCGGCAAGCAGGCGCT |
| | | GGAGACGGTCCAGCGGCTGTTGCCGGTGCTGTGCCAGGCCCACGGCTTGACCCCGGAGCAGGTGGTGG |
| | | CCATCGCCAGCCACGATGGCGGCAAGCAGGCGCTGGAGACGGTCCAGCGGCTGTTGCCGGTGCTGTGCC |
| | | AGGCCCACGGCTTGACCCCCCAGCAGGTGGTGGCCATCGCCAGCAATGGCGGTGGCAAGCAGGCGCTG |
| | | GAGACGGTCCAGCGGCTGTTGCCGGTGCTGTGCCAGGCCCACGGCTTGACCCCGGAGCAGGTGGTGGCC |
| | | ATCGCCAGCCACGATGGCGGCAAGCAGGCGCTGGAGACGGTCCAGCGGCTGTTGCCGGTGCTGTGCCAG |
| | | GCCCACGGCTTGACCCCGGAGCAGGTGGTGGCCATCGCCAGCCACGATGGCGGCAAGCAGGCGCTGGA |
| | | GACGGTCCAGCGGCTGTTGCCGGTGCTGTGCCAGGCCCACGGCTTGACCCCGGAGCAGGTGGTGGCCAT |
| | | CGCCAGCAATATTGGTGGCAAGCAGGCGCTGGAGACGGTGCAGGCGCTGTTGCCGGTGCTGTGCCAGG |
| | | CCCACGGCTTGACCCCCCAGCAGGTGGTGGCCATCGCCAGCAATAATGGTGGCAAGCAGGCGCTGGAGA |
| | | CGGTCCAGCGGCTGTTGCCGGTGCTGTGCCAGGCCCACGGCTTGACCCCCCAGCAGGTGGTGGCCATCG |
| | | CCAGCAATAATGGTGGCAAGCAGGCGCTGGAGACGGTCCAGCGGCTGTTGCCGGTGCTGTGCCAGGCCC |
| | | ACGGCTTGACCCCGGAGCAGGTGGTGGCCATCGCCAGCCACGATGGCGGCAAGCAGGCGCTGGAGACG |
| | | GTCCAGCGGCTGTTGCCGGTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCGGTGCTGTGCCAGGCCCA |
| | | CGGCTTGACCCCGGAGCAGGTGGTGGCCATCGCCAGCAATATTGGTGGCAAGCAGGCGCTGGAGACGG |
| | | TGCAGGCGCTGTTGCCGGTGCTGTGCCAGGCCCACGGCTTGACCCCCCAGCAGGTGGTGGCCATCGCCA |
| | | GCAATAATGGTGGCAAGCAGGCGCTGGAGACGGTCCAGCGGCTGTTGCCGGTGCTGTGCCAGGCCCACG |
| | | GCTTGACCCCGGAGCAGGTGGTGGCCATCGCCAGCAATATTGGTGGCAAGCAGGCGCTGGAGACGGTG |
| | | CAGGCGCTGTTGCCGGTGCTGTGCCAGGCCCACGGCTTGACCCCTCAGCAGGTGGTGGCCATCGCCAGC |
| | | AATGGCGGCGGCAGGCCGGCGCTGGAGAGCATTGTTGCCCAGTTATCTCGCCCTGATCCGGCGTTGGCC |
| | | GCGTTGACCAACGACCACCTCGTCGCCTTGGCCTGCCTCGGCGGGCGTCCTGCGCTGGATGCAGTGAAAA |
| | | AGGGATTGGGGGATCCTATCAGCCGTTCCCAGCTGGTGAAGTCCGAGCTGGAGGAGAAGAAATCCGAGT |
| | | TGAGGCACAAGCTGAAGTACGTGCCCCACGAGTACATCGAGCTGATCGAGATCGCCCGGAACAGCACCC |
| | | AGGACCGTATCCTGGAGATGAAGGTGATGGAGTTCTTCATGAAGGTGTACGGCTACAGGGGCAAGCACC |
| | | TGGGCGGCTCCAGGAAGCCCGACGGCGCCATCTACACCGTGGGCTCCCCCATCGACTACGGCGTGATCGT |
| | | GGACACCAAGGCCTACTCCGGCGGCTACAACCTGCCCATCGGCCAGGCCGACGAAATGCAGAGGTACGT |
| | | GGAGGAGAACCAGACCAGGAACAAGCACATCAACCCCAACGAGTGGTGGAAGGTGTACCCCTCCAGCGT |
| | | GACCGAGTTCAAGTTCCTGTTCGTGTCCGGCCACTTCAAGGGCAACTACAAGGCCCAGCTGACCAGGCTG |
| | | AACCACATCACCAACTGCAACGGCGCCGTGCTGTCCGTGGAGGAGCTCCTGATCGGCGGCGAGATGATC |
| | | AAGGCCGGCACCCTGACCCTGGAGGAGGTGAGGAGGAAGTTCAACAACGGCGAGATCAACTTCGCGGC |
| | | CGACTGATAA |
| T02 | 83 | TCCAAAGATTCAGGTTTACTCACGTCATCCAGCAGAGAATGGAAAGTCAA |
| Beta2M | | |
| target | | |
| T02 | 84 | ATGGGCGATCCTAAAAAGAAACGTAAGGTCATCGATTACCCATACGATGTTCCAGATTACGCTATCGATA |
| TALEN | | TCGCCGATCTACGCACGCTCGGCTACAGCCAGCAGCAACAGGAGAAGATCAAACCGAAGGTTCGTTCGA |
| Beta2M | | CAGTGGCGCAGCACCACGAGGCACTGGTCGGCCACGGGTTTACACACGCGCACATCGTTGCGTTAAGCC |
| LEFT | | AACACCCGGCAGCGTTAGGGACCGTCGCTGTCAAGTATCAGGACATGATCGCAGCGTTGCCAGAGGCGA |
| | | CACACGAAGCGATCGTTGGCGTCGGCAAACAGTGGTCCGGCGCACGCGCTCTGGAGGCCTTGCTCACGG |
| | | TGGCGGGAGAGTTGAGAGGTCCACCGTTACAGTTGGACACAGGCCAACTTCTCAAGATTGCAAAACGTG |
| | | GCGGCGTGACCGCAGTGGAGGCAGTGCATGCATGGCGCAATGCACTGACGGGTGCCCCGCTCAACTTGA |
| | | CCCCGGAGCAGGTGGTGGCCATCGCCAGCCACGATGGCGGCAAGCAGGCGCTGGAGACGGTCCAGCGG |
| | | CTGTTGCCGGTGCTGTGCCAGGCCCACGGCTTGACCCCGGAGCAGGTGGTGGCCATCGCCAGCCACGAT |
| | | GGCGGCAAGCAGGCGCTGGAGACGGTCCAGCGGCTGTTGCCGGTGCTGTGCCAGGCCCACGGCTTGAC |
| | | CCCGGAGCAGGTGGTGGCCATCGCCAGCAATATTGGTGGCAAGCAGGCGCTGGAGACGGTGCAGGCGC |
| | | TGTTGCCGGTGCTGTGCCAGGCCCACGGCTTGACCCCGGAGCAGGTGGTGGCCATCGCCAGCAATATTG |
| | | GTGGCAAGCAGGCGCTGGAGACGGTGCAGGCGCTGTTGCCGGTGCTGTGCCAGGCCCACGGCTTGACCC |
| | | CGGAGCAGGTGGTGGCCATCGCCAGCAATATTGGTGGCAAGCAGGCGCTGGAGACGGTGCAGGCGCTG |
| | | TTGCCGGTGCTGTGCCAGGCCCACGGCTTGACCCCCCAGCAGGTGGTGGCCATCGCCAGCAATAATGGT |
| | | GGCAAGCAGGCGCTGGAGACGGTCCAGCGGCTGTTGCCGGTGCTGTGCCAGGCCCACGGCTTGACCCCG |
| | | GAGCAGGTGGTGGCCATCGCCAGCAATATTGGTGGCAAGCAGGCGCTGGAGACGGTGCAGGCGCTGTT |
| | | GCCGGTGCTGTGCCAGGCCCACGGCTTGACCCCCCAGCAGGTGGTGGCCATCGCCAGCAATGGCGGTGG |
| | | CAAGCAGGCGCTGGAGACGGTCCAGCGGCTGTTGCCGGTGCTGTGCCAGGCCCACGGCTTGACCCCCCA |
| | | GCAGGTGGTGGCCATCGCCAGCAATGGCGGTGGCAAGCAGGCGCTGGAGACGGTCCAGCGGCTGTTGC |
| | | CGGTGCTGTGCCAGGCCCACGGCTTGACCCCGGAGCAGGTGGTGGCCATCGCCAGCCACGATGGCGGCA |
| | | AGCAGGCGCTGGAGACGGTCCAGCGGCTGTTGCCGGTGCTGTGCCAGGCCCACGGCTTGACCCCGGAGC |
| | | AGGTGGTGGCCATCGCCAGCAATATTGGTGGCAAGCAGGCGCTGGAGACGGTGCAGGCGCTGTTGCCG |
| | | GTGCTGTGCCAGGCCCACGGCTTGACCCCCCAGCAGGTGGTGGCCATCGCCAGCAATAATGGTGGCAAG |
| | | CAGGCGCTGGAGACGGTCCAGCGGCTGTTGCCGGTGCTGTGCCAGGCCCACGGCTTGACCCCCCAGCAG |
| | | GTGGTGGCCATCGCCAGCAATAATGGTGGCAAGCAGGCGCTGGAGACGGTCCAGCGGCTGTTGCCGGT |
| | | GCTGTGCCAGGCCCACGGCTTGACCCCCCAGCAGGTGGTGGCCATCGCCAGCAATGGCGGTGGCAAGCA |
| | | GGCGCTGGAGACGGTCCAGCGGCTGTTGCCGGTGCTGTGCCAGGCCCACGGCTTGACCCCCCAGCAGGT |
| | | GGTGGCCATCGCCAGCAATGGCGGTGGCAAGCAGGCGCTGGAGACGGTCCAGCGGCTGTTGCCGGTGC |
| | | TGTGCCAGGCCCACGGCTTGACCCCTCAGCAGGTGGTGGCCATCGCCAGCAATGGCGGCGGCAGGCCGG |
| | | CGCTGGAGAGCATTGTTGCCCAGTTATCTCGCCCTGATCCGGCGTTGGCCGCGTTGACCAACGACCACCT |
| | | CGTCGCCTTGGCCTGCCTCGGCGGGCGTCCTGCGCTGGATGCAGTGAAAAAGGGATTGGGGGATCCTAT |
| | | CAGCCGTTCCCAGCTGGTGAAGTCCGAGCTGGAGGAGAAGAAATCCGAGTTGAGGCACAAGCTGAAGTA |
| | | CGTGCCCCACGAGTACATCGAGCTGATCGAGATCGCCCGGAACAGCACCCAGGACCGTATCCTGGAGAT |
| | | GAAGGTGATGGAGTTCTTCATGAAGGTGTACGGCTACAGGGGCAAGCACCTGGGCGGCTCCAGGAAGC |
| | | CCGACGGCGCCATCTACACCGTGGGCTCCCCCATCGACTACGGCGTGATCGTGGACACCAAGGCCTACTC |
| | | CGGCGGCTACAACCTGCCCATCGGCCAGGCCGACGAAATGCAGAGGTACGTGGAGGAGAACCAGACCA |
| | | GGAACAAGCACATCAACCCCAACGAGTGGTGGAAGGTGTACCCCTCCAGCGTGACCGAGTTCAAGTTCCT |
| | | GTTCGTGTCCGGCCACTTCAAGGGCAACTACAAGGCCCAGCTGACCAGGCTGAACCACATCACCAACTGC |
| | | AACGGCGCCGTGCTGTCCGTGGAGGAGCTCCTGATCGGCGGCGAGATGATCAAGGCCGGCACCCTGACC |
| | | CTGGAGGAGGTGAGGAGGAAGTTCAACAACGGCGAGATCAACTTCGCGGCCGACTGATAA |
| T02 | 85 | ATGGGCGATCCTAAAAAGAAACGTAAGGTCATCGATAAGGAGACCGCCGCTGCCAAGTTCGAGAGACAG |
| TALEN | | CACATGGACAGCATCGATATCGCCGATCTACGCACGCTCGGCTACAGCCAGCAGCAACAGGAGAAGATC |
| Beta2M | | AAACCGAAGGTTCGTTCGACAGTGGCGCAGCACCACGAGGCACTGGTCGGCCACGGGTTTACACACGCG |
| RIGHT | | CACATCGTTGCGTTAAGCCAACACCCGGCAGCGTTAGGGACCGTCGCTGTCAAGTATCAGGACATGATCG |
| | | CAGCGTTGCCAGAGGCGACACACGAAGCGATCGTTGGCGTCGGCAAACAGTGGTCCGGCGCACGCGCTC |
| | | TGGAGGCCTTGCTCACGGTGGCGGGAGAGTTGAGAGGTCCACCGTTACAGTTGGACACAGGCCAACTTC |
| | | TCAAGATTGCAAAACGTGGCGGCGTGACCGCAGTGGAGGCAGTGCATGCATGGCGCAATGCACTGACG |
| | | GGTGCCCCGCTCAACTTGACCCCCCAGCAGGTGGTGGCCATCGCCAGCAATAATGGTGGCAAGCAGGCG |
| | | CTGGAGACGGTCCAGCGGCTGTTGCCGGTGCTGTGCCAGGCCCACGGCTTGACCCCGGAGCAGGTGGTG |
| | | GCCATCGCCAGCAATATTGGTGGCAAGCAGGCGCTGGAGACGGTGCAGGCGCTGTTGCCGGTGCTGTGC |
| | | CAGGCCCACGGCTTGACCCCGGAGCAGGTGGTGGCCATCGCCAGCCACGATGGCGGCAAGCAGGCGCT |
| | | GGAGACGGTCCAGCGGCTGTTGCCGGTGCTGTGCCAGGCCCACGGCTTGACCCCCCAGCAGGTGGTGGC |
| | | CATCGCCAGCAATGGCGGTGGCAAGCAGGCGCTGGAGACGGTCCAGCGGCTGTTGCCGGTGCTGTGCCA |
| | | GGCCCACGGCTTGACCCCCCAGCAGGTGGTGGCCATCGCCAGCAATGGCGGTGGCAAGCAGGCGCTGG |
| | | AGACGGTCCAGCGGCTGTTGCCGGTGCTGTGCCAGGCCCACGGCTTGACCCCCCAGCAGGTGGTGGCCA |
| | | TCGCCAGCAATGGCGGTGGCAAGCAGGCGCTGGAGACGGTCCAGCGGCTGTTGCCGGTGCTGTGCCAG |
| | | GCCCACGGCTTGACCCCGGAGCAGGTGGTGGCCATCGCCAGCCACGATGGCGGCAAGCAGGCGCTGGA |
| | | GACGGTCCAGCGGCTGTTGCCGGTGCTGTGCCAGGCCCACGGCTTGACCCCGGAGCAGGTGGTGGCCAT |
| | | CGCCAGCCACGATGGCGGCAAGCAGGCGCTGGAGACGGTCCAGCGGCTGTTGCCGGTGCTGTGCCAGG |
| | | CCCACGGCTTGACCCCGGAGCAGGTGGTGGCCATCGCCAGCAATATTGGTGGCAAGCAGGCGCTGGAGA |
| | | CGGTGCAGGCGCTGTTGCCGGTGCTGTGCCAGGCCCACGGCTTGACCCCCCAGCAGGTGGTGGCCATCG |
| | | CCAGCAATGGCGGTGGCAAGCAGGCGCTGGAGACGGTCCAGCGGCTGTTGCCGGTGCTGTGCCAGGCC |
| | | CACGGCTTGACCCCCCAGCAGGTGGTGGCCATCGCCAGCAATGGCGGTGGCAAGCAGGCGCTGGAGAC |
| | | GGTCCAGCGGCTGTTGCCGGTGCTGTGCCAGGCCCACGGCTTGACCCCGGAGCAGGTGGTGGCCATCGC |
| | | CAGCCACGATGGCGGCAAGCAGGCGCTGGAGACGGTCCAGCGGCTGTTGCCGGTGCTGTGCCAGGCCC |
| | | ACGGCTTGACCCCCCAGCAGGTGGTGGCCATCGCCAGCAATGGCGGTGGCAAGCAGGCGCTGGAGACG |
| | | GTCCAGCGGCTGTTGCCGGTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCGGTGCTGTGCCAGGCCCA |
| | | CGGCTTGACCCCCCAGCAGGTGGTGGCCATCGCCAGCAATGGCGGTGGCAAGCAGGCGCTGGAGACGG |
| | | TCCAGCGGCTGTTGCCGGTGCTGTGCCAGGCCCACGGCTTGACCCCTCAGCAGGTGGTGGCCATCGCCAG |
| | | CAATGGCGGCGGCAGGCCGGCGCTGGAGAGCATTGTTGCCCAGTTATCTCGCCCTGATCCGGCGTTGGC |
| | | CGCGTTGACCAACGACCACCTCGTCGCCTTGGCCTGCCTCGGCGGGCGTCCTGCGCTGGATGCAGTGAAA |
| | | AAGGGATTGGGGGATCCTATCAGCCGTTCCCAGCTGGTGAAGTCCGAGCTGGAGGAGAAGAAATCCGA |
| | | GTTGAGGCACAAGCTGAAGTACGTGCCCCACGAGTACATCGAGCTGATCGAGATCGCCCGGAACAGCAC |
| | | CCAGGACCGTATCCTGGAGATGAAGGTGATGGAGTTCTTCATGAAGGTGTACGGCTACAGGGGCAAGCA |
| | | CCTGGGCGGCTCCAGGAAGCCCGACGGCGCCATCTACACCGTGGGCTCCCCCATCGACTACGGCGTGATC |
| | | GTGGACACCAAGGCCTACTCCGGCGGCTACAACCTGCCCATCGGCCAGGCCGACGAAATGCAGAGGTAC |
| | | GTGGAGGAGAACCAGACCAGGAACAAGCACATCAACCCCAACGAGTGGTGGAAGGTGTACCCCTCCAGC |
| | | GTGACCGAGTTCAAGTTCCTGTTCGTGTCCGGCCACTTCAAGGGCAACTACAAGGCCCAGCTGACCAGGC |
| | | TGAACCACATCACCAACTGCAACGGCGCCGTGCTGTCCGTGGAGGAGCTCCTGATCGGCGGCGAGATGA |
| | | TCAAGGCCGGCACCCTGACCCTGGAGGAGGTGAGGAGGAAGTTCAACAACGGCGAGATCAACTTCGCG |
| | | GCCGACTGATAA |
| T03 | 86 | TTAGCTGTGCTCGCGCTACTCTCTCTTTCTGGCCTGGAGGCTATCCA |
| Beta2M | | |
| target | | |
| T03 | 87 | ATGGGCGATCCTAAAAAGAAACGTAAGGTCATCGATTACCCATACGATGTTCCAGATTACGCTATCGATA |
| TALEN | | TCGCCGATCTACGCACGCTCGGCTACAGCCAGCAGCAACAGGAGAAGATCAAACCGAAGGTTCGTTCGA |
| Beta2M | | CAGTGGCGCAGCACCACGAGGCACTGGTCGGCCACGGGTTTACACACGCGCACATCGTTGCGTTAAGCC |
| LEFT | | AACACCCGGCAGCGTTAGGGACCGTCGCTGTCAAGTATCAGGACATGATCGCAGCGTTGCCAGAGGCGA |
| | | CACACGAAGCGATCGTTGGCGTCGGCAAACAGTGGTCCGGCGCACGCGCTCTGGAGGCCTTGCTCACGG |
| | | TGGCGGGAGAGTTGAGAGGTCCACCGTTACAGTTGGACACAGGCCAACTTCTCAAGATTGCAAAACGTG |
| | | GCGGCGTGACCGCAGTGGAGGCAGTGCATGCATGGCGCAATGCACTGACGGGTGCCCCGCTCAACTTGA |
| | | CCCCGGAGCAGGTGGTGGCCATCGCCAGCAATATTGGTGGCAAGCAGGCGCTGGAGACGGTGCAGGCG |
| | | CTGTTGCCGGTGCTGTGCCAGGCCCACGGCTTGACCCCCCAGCAGGTGGTGGCCATCGCCAGCAATAATG |
| | | GTGGCAAGCAGGCGCTGGAGACGGTCCAGCGGCTGTTGCCGGTGCTGTGCCAGGCCCACGGCTTGACCC |
| | | CGGAGCAGGTGGTGGCCATCGCCAGCCACGATGGCGGCAAGCAGGCGCTGGAGACGGTCCAGCGGCTG |
| | | TTGCCGGTGCTGTGCCAGGCCCACGGCTTGACCCCCCAGCAGGTGGTGGCCATCGCCAGCAATGGCGGT |
| | | GGCAAGCAGGCGCTGGAGACGGTCCAGCGGCTGTTGCCGGTGCTGTGCCAGGCCCACGGCTTGACCCCC |
| | | CAGCAGGTGGTGGCCATCGCCAGCAATAATGGTGGCAAGCAGGCGCTGGAGACGGTCCAGCGGCTGTT |
| | | GCCGGTGCTGTGCCAGGCCCACGGCTTGACCCCCCAGCAGGTGGTGGCCATCGCCAGCAATGGCGGTGG |
| | | CAAGCAGGCGCTGGAGACGGTCCAGCGGCTGTTGCCGGTGCTGTGCCAGGCCCACGGCTTGACCCCCCA |
| | | GCAGGTGGTGGCCATCGCCAGCAATAATGGTGGCAAGCAGGCGCTGGAGACGGTCCAGCGGCTGTTGC |
| | | CGGTGCTGTGCCAGGCCCACGGCTTGACCCCGGAGCAGGTGGTGGCCATCGCCAGCCACGATGGCGGCA |
| | | AGCAGGCGCTGGAGACGGTCCAGCGGCTGTTGCCGGTGCTGTGCCAGGCCCACGGCTTGACCCCCCAGC |
| | | AGGTGGTGGCCATCGCCAGCAATGGCGGTGGCAAGCAGGCGCTGGAGACGGTCCAGCGGCTGTTGCCG |
| | | GTGCTGTGCCAGGCCCACGGCTTGACCCCGGAGCAGGTGGTGGCCATCGCCAGCCACGATGGCGGCAAG |
| | | CAGGCGCTGGAGACGGTCCAGCGGCTGTTGCCGGTGCTGTGCCAGGCCCACGGCTTGACCCCCCAGCAG |
| | | GTGGTGGCCATCGCCAGCAATAATGGTGGCAAGCAGGCGCTGGAGACGGTCCAGCGGCTGTTGCCGGT |
| | | GCTGTGCCAGGCCCACGGCTTGACCCCGGAGCAGGTGGTGGCCATCGCCAGCCACGATGGCGGCAAGCA |
| | | GGCGCTGGAGACGGTCCAGCGGCTGTTGCCGGTGCTGTGCCAGGCCCACGGCTTGACCCCCCAGCAGGT |
| | | GGTGGCCATCGCCAGCAATAATGGTGGCAAGCAGGCGCTGGAGACGGTCCAGCGGCTGTTGCCGGTGCT |
| | | GTGCCAGGCCCACGGCTTGACCCCGGAGCAGGTGGTGGCCATCGCCAGCCACGATGGCGGCAAGCAGG |
| | | CGCTGGAGACGGTCCAGCGGCTGTTGCCGGTGCTGTGCCAGGCCCACGGCTTGACCCCCCAGCAGGTGG |
| | | TGGCCATCGCCAGCAATGGCGGTGGCAAGCAGGCGCTGGAGACGGTCCAGCGGCTGTTGCCGGTGCTGT |
| | | GCCAGGCCCACGGCTTGACCCCTCAGCAGGTGGTGGCCATCGCCAGCAATGGCGGCGGCAGGCCGGCGC |
| | | TGGAGAGCATTGTTGCCCAGTTATCTCGCCCTGATCCGGCGTTGGCCGCGTTGACCAACGACCACCTCGTC |
| | | GCCTTGGCCTGCCTCGGCGGGCGTCCTGCGCTGGATGCAGTGAAAAAGGGATTGGGGGATCCTATCAGC |
| | | CGTTCCCAGCTGGTGAAGTCCGAGCTGGAGGAGAAGAAATCCGAGTTGAGGCACAAGCTGAAGTACGTG |
| | | CCCCACGAGTACATCGAGCTGATCGAGATCGCCCGGAACAGCACCCAGGACCGTATCCTGGAGATGAAG |
| | | GTGATGGAGTTCTTCATGAAGGTGTACGGCTACAGGGGCAAGCACCTGGGCGGCTCCAGGAAGCCCGAC |
| | | GGCGCCATCTACACCGTGGGCTCCCCCATCGACTACGGCGTGATCGTGGACACCAAGGCCTACTCCGGCG |
| | | GCTACAACCTGCCCATCGGCCAGGCCGACGAAATGCAGAGGTACGTGGAGGAGAACCAGACCAGGAAC |
| | | AAGCACATCAACCCCAACGAGTGGTGGAAGGTGTACCCCTCCAGCGTGACCGAGTTCAAGTTCCTGTTCG |
| | | TGTCCGGCCACTTCAAGGGCAACTACAAGGCCCAGCTGACCAGGCTGAACCACATCACCAACTGCAACGG |
| | | CGCCGTGCTGTCCGTGGAGGAGCTCCTGATCGGCGGCGAGATGATCAAGGCCGGCACCCTGACCCTGGA |
| | | GGAGGTGAGGAGGAAGTTCAACAACGGCGAGATCAACTTCGCGGCCGACTGATAA |
| T03 | 88 | ATGGGCGATCCTAAAAAGAAACGTAAGGTCATCGATAAGGAGACCGCCGCTGCCAAGTTCGAGAGACAG |
| TALEN | | CACATGGACAGCATCGATATCGCCGATCTACGCACGCTCGGCTACAGCCAGCAGCAACAGGAGAAGATC |
| Beta2M | | AAACCGAAGGTTCGTTCGACAGTGGCGCAGCACCACGAGGCACTGGTCGGCCACGGGTTTACACACGCG |
| RIGHT | | CACATCGTTGCGTTAAGCCAACACCCGGCAGCGTTAGGGACCGTCGCTGTCAAGTATCAGGACATGATCG |
| | | CAGCGTTGCCAGAGGCGACACACGAAGCGATCGTTGGCGTCGGCAAACAGTGGTCCGGCGCACGCGCTC |
| | | TGGAGGCCTTGCTCACGGTGGCGGGAGAGTTGAGAGGTCCACCGTTACAGTTGGACACAGGCCAACTTC |
| | | TCAAGATTGCAAAACGTGGCGGCGTGACCGCAGTGGAGGCAGTGCATGCATGGCGCAATGCACTGACG |
| | | GGTGCCCCGCTCAACTTGACCCCCCAGCAGGTGGTGGCCATCGCCAGCAATAATGGTGGCAAGCAGGCG |
| | | CTGGAGACGGTCCAGCGGCTGTTGCCGGTGCTGTGCCAGGCCCACGGCTTGACCCCCCAGCAGGTGGTG |
| | | GCCATCGCCAGCAATAATGGTGGCAAGCAGGCGCTGGAGACGGTCCAGCGGCTGTTGCCGGTGCTGTGC |
| | | CAGGCCCACGGCTTGACCCCGGAGCAGGTGGTGGCCATCGCCAGCAATATTGGTGGCAAGCAGGCGCTG |
| | | GAGACGGTGCAGGCGCTGTTGCCGGTGCTGTGCCAGGCCCACGGCTTGACCCCCCAGCAGGTGGTGGCC |
| | | ATCGCCAGCAATGGCGGTGGCAAGCAGGCGCTGGAGACGGTCCAGCGGCTGTTGCCGGTGCTGTGCCA |
| | | GGCCCACGGCTTGACCCCGGAGCAGGTGGTGGCCATCGCCAGCAATATTGGTGGCAAGCAGGCGCTGGA |
| | | GACGGTGCAGGCGCTGTTGCCGGTGCTGTGCCAGGCCCACGGCTTGACCCCCCAGCAGGTGGTGGCCAT |
| | | CGCCAGCAATAATGGTGGCAAGCAGGCGCTGGAGACGGTCCAGCGGCTGTTGCCGGTGCTGTGCCAGGC |
| | | CCACGGCTTGACCCCGGAGCAGGTGGTGGCCATCGCCAGCCACGATGGCGGCAAGCAGGCGCTGGAGA |
| | | CGGTCCAGCGGCTGTTGCCGGTGCTGTGCCAGGCCCACGGCTTGACCCCGGAGCAGGTGGTGGCCATCG |
| | | CCAGCCACGATGGCGGCAAGCAGGCGCTGGAGACGGTCCAGCGGCTGTTGCCGGTGCTGTGCCAGGCCC |
| | | ACGGCTTGACCCCCCAGCAGGTGGTGGCCATCGCCAGCAATGGCGGTGGCAAGCAGGCGCTGGAGACG |
| | | GTCCAGCGGCTGTTGCCGGTGCTGTGCCAGGCCCACGGCTTGACCCCGGAGCAGGTGGTGGCCATCGCC |
| | | AGCCACGATGGCGGCAAGCAGGCGCTGGAGACGGTCCAGCGGCTGTTGCCGGTGCTGTGCCAGGCCCA |
| | | CGGCTTGACCCCGGAGCAGGTGGTGGCCATCGCCAGCCACGATGGCGGCAAGCAGGCGCTGGAGACGG |
| | | TCCAGCGGCTGTTGCCGGTGCTGTGCCAGGCCCACGGCTTGACCCCGGAGCAGGTGGTGGCCATCGCCA |
| | | GCAATATTGGTGGCAAGCAGGCGCTGGAGACGGTGCAGGCGCTGTTGCCGGTGCTGTGCCAGGCCCAC |
| | | GGCTTGACCCCCCAGCAGGTGGTGGCCATCGCCAGCAATAATGGTGGCAAGCAGGCGCTGGAGACGGTC |
| | | CAGCGGCTGTTGCCGGTGCTGTGCCAGGCCCACGGCTTGACCCCCCAGCAGGTGGTGGCCATCGCCAGC |
| | | AATAATGGTGGCAAGCAGGCGCTGGAGACGGTCCAGCGGCTGTTGCCGGTGCTGTGCCAGGCCCACGGC |
| | | TTGACCCCGGAGCAGGTGGTGGCCATCGCCAGCCACGATGGCGGCAAGCAGGCGCTGGAGACGGTCCA |
| | | GCGGCTGTTGCCGGTGCTGTGCCAGGCCCACGGCTTGACCCCTCAGCAGGTGGTGGCCATCGCCAGCAA |
| | | TGGCGGCGGCAGGCCGGCGCTGGAGAGCATTGTTGCCCAGTTATCTCGCCCTGATCCGGCGTTGGCCGC |
| | | GTTGACCAACGACCACCTCGTCGCCTTGGCCTGCCTCGGCGGGCGTCCTGCGCTGGATGCAGTGAAAAAG |
| | | GGATTGGGGGATCCTATCAGCCGTTCCCAGCTGGTGAAGTCCGAGCTGGAGGAGAAGAAATCCGAGTTG |
| | | AGGCACAAGCTGAAGTACGTGCCCCACGAGTACATCGAGCTGATCGAGATCGCCCGGAACAGCACCCAG |
| | | GACCGTATCCTGGAGATGAAGGTGATGGAGTTCTTCATGAAGGTGTACGGCTACAGGGGCAAGCACCTG |
| | | GGCGGCTCCAGGAAGCCCGACGGCGCCATCTACACCGTGGGCTCCCCCATCGACTACGGCGTGATCGTG |
| | | GACACCAAGGCCTACTCCGGCGGCTACAACCTGCCCATCGGCCAGGCCGACGAAATGCAGAGGTACGTG |
| | | GAGGAGAACCAGACCAGGAACAAGCACATCAACCCCAACGAGTGGTGGAAGGTGTACCCCTCCAGCGTG |
| | | ACCGAGTTCAAGTTCCTGTTCGTGTCCGGCCACTTCAAGGGCAACTACAAGGCCCAGCTGACCAGGCTGA |
| | | ACCACATCACCAACTGCAACGGCGCCGTGCTGTCCGTGGAGGAGCTCCTGATCGGCGGCGAGATGATCA |
| | | AGGCCGGCACCCTGACCCTGGAGGAGGTGAGGAGGAAGTTCAACAACGGCGAGATCAACTTCGCGGCC |
| | | GACTGATAA |

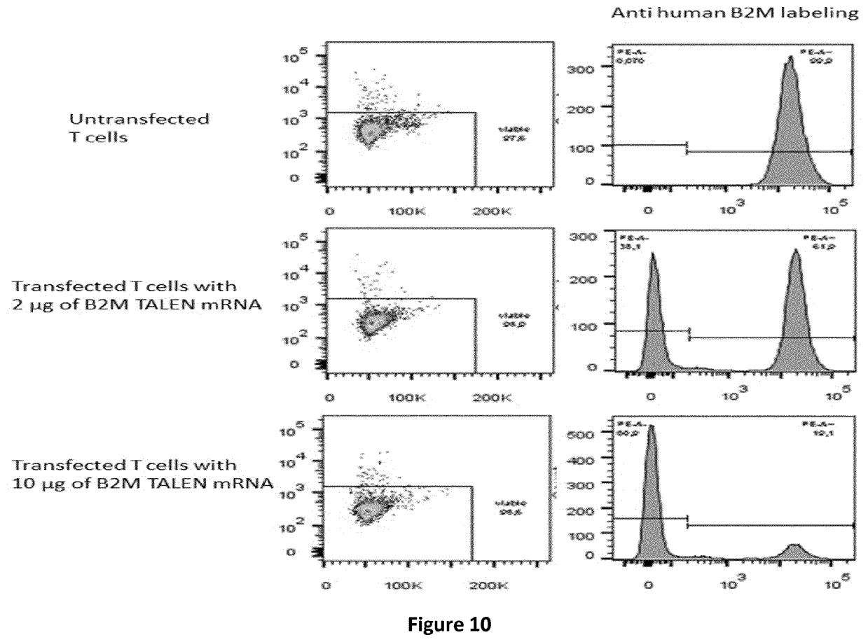

To test the ability of this β2M specific TALEN to promote error-prone NHEJ events at the β2M locus, 2 or 10 μg of mRNA encoding TALEN were electroporated in Primary T cells using Pulse Agile technology according to the manufacturer protocol. Three days post transfection, cells were recovered and labeled with a specific β2-microglobulin antibody coupled to the PhycoErythrin fluorochrome. Cells are then analyzed by flow cytometry for viability and β2-m expression. The results are shown on FIG. 10. On the top panel, nearly 100% of untransfected T cells express β2-m (top right panel). Transfection of T cells with the specific β2M TALEN reduces dramatically [2-m expression since 38% (middle right) and 80% of T cells (bottom right panel) become beta2-m negative when transfected with 2 μg or 10 μg of TALEN mRNA respectively. These data indicates that β2M knock-out in T cells can be achieved with high efficacy.

### Production and Expression of the Single Chain Molecule β2M-UL18 in T Cells

HCMV UL18 encodes a type I transmembrane glycoprotein that shares a high level of AA sequence identity with MHC Class I molecules that associates with beta2-m and binds endogenous peptides. Since our goal is to express this molecule in T cells where β2M gene has been invalidated, our strategy is to produce a chimeric molecule where beta2-m and UL18 is fused as a single chain polypeptide. SEQ ID N°89 shows the amino-acid sequence of the chimeric protein. Lentiviral particles containing the chimeric β2M-UL18 are transduced into T cells. Expression of transgene is monitored by FACS analysis using a beta2-m antibody. The results from this experiment aim to show that a β2M-UL18 chimeric protein is efficiently expressed in T cells.

### Production and Expression of NKG2D Ligands in T Cells

NKG2D natural ligands are transmembrane or GPI-anchored proteins. In order to achieve secretion of these molecules by T cells, the extra-cellular domains of NKG2D ligands have been fused in their N-terminus to a secretory peptide form. Amino-acid sequences of secreted chimeric NKG2D ligands are listed below (SEQ ID NO:90 to SEQ ID NO:97). Lentiviral particles containing the chimeric NKG2D ligands are transduced into T cells. Expression of transgene in culture supernatant is monitored by Western Blot analysis using specific antibodies. The results from this experiment aim to show that chimeric NKG2D ligand proteins are efficiently expressed in T cells.

### Beta2-M Deficient CAR T Cells are not Recognized by Allogenic T Cells.

PBMCs from healthy donor A is co-cultured with irradiated or mitomycin-treated engineered beta2-m deficient T cells from donor B. As a control, PBMCs from healthy donor A is co-cultured with irradiated or mitomycin-treated engineered beta2-m positive T cells from donor B. 7 days later, cells proliferation from donor A is measured by XTT colorimetric assay or by CFSE dilution (FACS analysis). Although cell proliferation is observed in control, no or limited cell proliferation is observed when engineered T cells do not express beta2-m. The results from this experiment aim to show that alloreactive T cells are not able to recognize and proliferate against beta2-m deficient T cells.

### Efficient Inhibition of NK Mediated Engineered T Cells Lysis

NK cells are purified from healthy donor A PBMCs. As targets, engineered T cells from healthy donor B are produced and listed below. a) engineered T cells (negative control), b) beta2-m deficient engineered T cells (positive control), c) beta2-m deficient engineered T cells expressing β2M-UL18 (SEQ ID N° 89), d-k) beta2-m deficient engineered T cells expressing respectively SP-MICAed (SEQ ID N° 90), SP-MICBed (SEQ ID N°91), SP-ULBP1ed (SEQ ID N° 92), SP-ULBP2ed (SEQ ID N° 93), SP-ULBP3ed (SEQ ID N°94), SP-N2DL4ed (SEQID N° 95), SP-RET1Ged (SEQ ID N°96), SP-RAETILed (SEQ ID N°97). These sequences are reported in the following Table 10.

TABLE 10
Polypeptide sequence of a viral MHC homolog (UL18) and a panel of NKG2D
ligands to be expressed according to the present invention.

| | SEQ | |
|---|---|---|
| | ID NO: | Polypeptide sequence |
| Chimeric | 89 | MALPVTALLLPLALLLHAARPSRSVALAVLALLSLSGLEAIQRTPKIQVYSRHPAENGKSNFLNCYVSG |
| B2M-UL18 | | FHPSDIEVDLLKNGERIEKVEHSDLSFSKDWSFYLLYYTEFTPTEKDEYACRVNHVTLSQPKIVKWDRD |
| | | MGGGGSGGGGSGGGGSGGGGSMTMWCLTLFVLWMLRVVGMHVLRYGYTGIFDDTSHMTLTVVGIFDGQH |
| | | FFTYHVNSSDKASSRANGTISWMANVSAAYPTYLDGERAKGDLIFNQTEQNLLELEIALGYRSQSVLTW |
| | | THECNTTENGSFVAGYEGFGWDGETLMELKDNLTLWTGPNYEISWLKQNKTYIDGKIKNISEGDTTIQR |
| | | NYLKGNCTQWSVIYSGFQTPVTHPVVKGGVRNQNDNRAEAFCTSYGFFPGEINITFIHYGNKAPDDSEP |
| | | QCNPLLPTFDGTFHQGCYVAIFCNQNYTCRVTHGNWTVEIPISVTSPDDSSSGEVPDHPTANKRYNTMT |
| | | ISSVLLALLLCALLFAFLHYFTTLKQYLRNLAFAWRYRKVRSS |
| SP-MICAed | 90 | MGGVLLTQRTLLSLVLALLFPSMASMEPHSLRYNLTVLSWDGSVQSGFLTEVHLDGQPFLRCDRQKCRA |
| | | KPQGQWAEDVLGNKTWDRETRDLTGNGKDLRMTLAHIKDQKEGLHSLQEIRVCEIHEDNSTRSSQHFYY |
| | | DGELFLSQNLETKEWTMPQSSRAQTLAMNVRNFLKEDAMKTKTHYHAMHADCLQELRRYLKSGVVLRRT |
| | | VPPMVNVTRSEASEGNITVTCRASGFYPWNITLSWRQDGVSLSHDTQQWGDVLPDGNGTYQTWVATRIC |
| | | QGEEQRFTCYMEHSGNHSTHPVPSGKVLVLQSHW |
| SP-MICBed | 91 | MGGVLLTQRTLLSLVLALLFPSMASMAEPHSLRYNLMVLSQDESVQSGFLAEGHLDGQPFLRYDRQKRR |
| | | AKPQGQWAEDVLGAKTWDTETEDLTENGQDLRRTLTHIKDQKGGLHSLQEIRVCEIHEDSSTRGSRHFY |
| | | YDGELFLSQNLETQESTVPQSSRAQTLAMNVTNFWKEDAMKTKTHYRAMQADCLQKLQRYLKSGVAIRR |
| | | TVPPMVNVTCSEVSEGNITVTCRASSFYPRNITLTWRQDGVSLSHNTQQWGDVLPDGNGTYQTWVATRI |
| | | RQGEEQRFTCYMEHSGNHGTHPVPSGKVLVLQSQRTD |
| SP-ULBP1ed | 92 | MGGVLLTQRTLLSLVLALLFPSMASMGWVDTHCLCYDFIITPKSRPEPQWCEVQGLVDERPFLHYDCVN |
| | | HKAKAFASLGKKVNVTKTWEEQTETLRDVVDFLKGQLLDIQVENLIPIEPLTLQARMSCEHEAHGHGRG |
| | | SWQFLFNGQKFLLFDSNNRKWTALHPGAKKMTEKWEKNRDVTMFFQKISLGDCKMWLEEFLMYWEQMLD |
| | | PT |
| SP-ULBP2ed | 93 | MGGVLLTQRTLLSLVLALLFPSMASMGRADPHSLCYDITVIPKFRPGPRWCAVQGQVDEKTFLHYDCGN |
| | | KTVTPVSPLGKKLNVTTAWKAQNPVLREVVDILTEQLRDIQLENYTPKEPLTLQARMSCEQKAEGHSSG |
| | | SWQFSFDGQIFLLFDSEKRMWTTVHPGARKMKEKWENDKVVAMSFHYFSMGDCIGWLEDFLMGMDSTLE |
| | | PSAG |
| SP-ULBP3ed | 94 | MGGVLLTQRTLLSLVLALLFPSMASMDAHSLWYNFTIIHLPRHGQQWCEVQSQVDQKNFLSYDCGSDKV |
| | | LSMGHLEEQLYATDAWGKQLEMLREVGQRLRLELADTELEDFTPSGPLTLQVRMSCECEADGYIRGSWQ |
| | | FSFDGRKFLLFDSNNRKWTWVHAGARRMKEKWEKDSGLTTFFKMVSMRDCKSWLRDFLMHRKKRLEPT |
| SP-N2DL4ed | 95 | MGGVLLTQRTLLSLVLALLFPSMASMHSLCFNFTIKSLSRPGQPWCEAQVFLNKNLFLQYNSDNNMVKP |
| | | LGLLGKKVYATSTWGELTQTLGEVGRDLRMLLCDIKPQIKTSDPSTLQVEMFCQREAERCTGASWQFAT |
| | | NGEKSLLFDAMNMTWTVINHEASKIKETWKKDRGLEKYFRKLSKGDCDHWLREFLGHWEAMPEPTVSPV |
| | | NASDIHWSSSSLPD |
| SP-RET1Ged | 96 | MGGVLLTQRTLLSLVLALLFPSMASMGLADPHSLCYDITVIPKFRPGPRWCAVQGQVDEKTFLHYDCGS |
| | | KTVTPVSPLGKKLNVTTAWKAQNPVLREVVDILTEQLLDIQLENYIPKEPLTLQARMSCEQKAEGHGSG |
| | | SWQLSFDGQIFLLFDSENRMWTTVHPGARKMKEKWENDKDMTMSFHYISMGDCTGWLEDFLMGMDSTLE |
| | | PSAGAPPTMSSGTAQPR |
| SP-RAETILed | 97 | MGGVLLTQRTLLSLVLALLFPSMASMRRDDPHSLCYDITVIPKFRPGPRWCAVQGQVDEKTFLHYDCGN |
| | | KTVTPVSPLGKKLNVTMAWKAQNPVLREVVDILTEQLLDIQLENYTPKEPLTLQARMSCEQKAEGHSSG |
| | | SWQFSIDGQTFLLFDSEKRMWTTVHPGARKMKEKWENDKDVAMSFHYISMGDCIGWLEDFLMGMDSTLE |
| | | PSAG |

Cytotoxicity mediated by NK cells was determined by a CFSE labeling assay. Target cells were labeled with CFSE, washed in PBS, mixed with NK cells at various E: T cell ratios and incubated for 4 h at 37° C. Cells are then analysed by flow cytometry and percentages of CFSE positive engineered T cells are measured, indicating the survival of engineered T cells in the presence of NK cells. It is intended that although NK mediated cell lysis is observed in the positive control (beta2-m deficient engineered T cells), no or limited NK mediated cell lysis is observed when beta2-m deficient engineered T cells engineered T cells express β2M-UL18 (SEQ ID N° 89) or secreted NKG2D ligands (SP-MICAed (SEQ ID N° 90), SP-MICBed (SEQ ID N° 91), SP-ULBP1ed (SEQ ID N°92), SP-ULBP2ed (SEQ ID N°93), SP-ULBP3ed (SEQ ID N°94), SP-N2DL4ed (SEQ ID N°95), SP-RET1Ged (SEQ ID N° 96), SP-RAETILed (SEQ ID N° 97). The results from this experiment aim to show that allogenic NK cells cytotoxicity activity is impaired when chimeric molecules, express in engineered T cells, act as decoy either for inhibitory signal receptor (β2M-UL18) or for stimulatory signal receptor (NKG2D ligands).

### List of References Cited in the Description

Ashwell, J. D. and R. D. Klusner (1990). “Genetic and mutational analysis of the T-cell antigen receptor.” Annu Rev Immunol 8:139-67.
Betts, M. R., J. M. Brenchley, et al. (2003). “Sensitive and viable identification of antigen-specific CD8+ T cells by a flow cytometric assay for degranulation.” J Immunol Methods 281 (1-2): 65-78.
Bierer B. E. et al. (1993) “Cyclosporin A and FK506: molecular mechanisms of immunosuppression and probes for transplantation biology.” Curr Opin Immunol 5 (5): 763-73.
Bix M. et al (1991). “Rejection of class I MHC-deficient haemopoietic cells by irradiated MHC-matched mice.” Nature 349 (6307): 329-31.
Boch, J., H. Scholze, et al. (2009). “Breaking the code of DNA binding specificity of TAL-type III effectors.” Science 326 (5959): 1509-12.
Cambier, J. C. (1995). “Antigen and Fc receptor signaling. The awesome power of the immunoreceptor tyrosine-based activation motif (ITAM).” J Immunol 155 (7): 3281-5.
Carter L, et al. (2002). “PD-1: PD-L inhibitory pathway affects both CD4 (+) and CD8 (+) T cells and is overcome by IL-2”. Eur. J. Immunol. 32 (3): 634-43.
Cong, L., F. A. Ran, et al. (2013). “Multiplex genome engineering using CRISPR/Cas systems.” Science 339 (6121): 819-23.
Critchlow, S. E. and S. P. Jackson (1998). “DNA end-joining: from yeast to man.” Trends Biochem Sci 23 (10): 394-8.
Deltcheva, E., K. Chylinski, et al. (2011). “CRISPR RNA maturation by trans-encoded small RNA and host factor RNase III.” Nature 471 (7340): 602-7.
Gasiunas, G. et al. (2012). “Cas9-crRNA ribonucleoprotein complex mediates specific DNA cleavage for adaptive immunity in bacteria.” Proc Natl Acad Sci USA 109 (39): E2579-86.
Haseloff and Gerlach (1988). “Simple RNA enzymes with new and highly specific endoribonuclease activities.” Nature 334:585-591.
Jena, B., G. Dotti, et al. (2010). “Redirecting T-cell specificity by introducing a tumor-specific chimeric antigen receptor.” Blood 116 (7): 1035-44.
Jinek, M., K. Chylinski, et al. (2012). “A programmable dual-RNA-guided DNA endonuclease in adaptive bacterial immunity.” Science 337 (6096): 816-21.
Liu L. et al. (1991). “Calcineurin is a common target of cyclophilin-cyclosporin A and FKBP-FK506 complexes.” Cell 66 (4): 807-15.
Ma, J. L., E. M. Kim, et al. (2003). “Yeast Mre11 and Rad1 proteins define a Ku-independent mechanism to repair double-strand breaks lacking overlapping end sequences.” Mol Cell Biol 23 (23): 8820-8.
Mach B., Steimle V, Reith W (1994). “MHC class II-deficient combined immunodeficiency: a disease of gene regulation”. Immunol. Rev. 138 (1): 207-21.
Mali, P., L. Yang, et al. (2013). “RNA-guided human genome engineering via Cas9.” Science 339 (6121): 823-6.
Moscou, M. J. and A. J. Bogdanove (2009). “A simple cipher governs DNA recognition by TAL effectors.” Science 326 (5959): 1501.
Park, T. S., S. A. Rosenberg, et al. (2011). “Treating cancer with genetically engineered T cells.” Trends Biotechnol 29 (11): 550-7.
Stoddard, B. L. (2005). “Homing endonuclease structure and function.” Q Rev Biophys 38 (1): 49-95.
Urnov F. D. et al. (2010) “Genome editing with engineered zinc finger nucleases” Nature reviews Genetics 11:636-646

1-18. (canceled)

19. A method for treating cancers, infections or immune disease in a patient comprising administering to said patient a population of allogeneic engineered T-cells, wherein at least 37% of said engineered T-cells have lost surface expression of HLA-A, HLA-B, and HLA-C by a genome inactivation of at least one gene encoding β2M and/or CIITA, and wherein expression of at least one gene encoding a component of TCR has been inactivated to make them non-alloreactive.

20. The method according to claim 1, wherein said population of allogeneic engineered T-cells is from a donor.

21. The method of claim 19, comprising a genome inactivation of β2M.

22. The method of claim 19, comprising a genome inactivation of CIITA.

23. The method according to claim 19, wherein said method is for treating solid tumors.

24. The method according to claim 19, wherein said pcancer is a carcinoma, melanoma, blastoma, sarcoma, leukemia or lymphoid malignancy.

25. The method according to claim 19, wherein said engineered T-cells are administered to said patient before, simultaneously or following bone marrow transplantation or T cell ablative therapy using either chemotherapy agents, external-beam radiation therapy (XRT), cyclophosphamide, or antibodies.

26. The method according to claim 19, wherein said engineered T-cells express a CAR.

27. The method according to claim 19, wherein the genes encoding β2M or CIITA and gene encoding a component of TCR have been inactivated in the T cells by cleavage using a rare-cutting endonuclease.

28. The method according to claim 27, wherein said rare-cutting endonuclease is TALE-nuclease, meganuclease, zing-finger nuclease (ZFN), or RNA guided endonuclease.

29. The method according to claim 27, wherein said rare-cutting endonuclease is TALE-nuclease.


SEQUENCE LISTING (97 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - DNA, 6673 bp, Homo sapiens
  source 1..6673: mol_type = unassigned DNA
aatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 3 - DNA, 987 bp, Homo sapiens
  source 1..987: mol_type = unassigned DNA
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 4 - protein, 1130 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 5 - DNA, 4654 bp, Homo sapiens
  source 1..4654: mol_type = unassigned DNA
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 6 - protein, 495 aa, synthetic construct
  REGION 1..495: note = anti-CD19 Cimeric Antigen Receptor
METDTLLLWVLLLWVPGSTGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 17 aa, synthetic construct
  REGION 1..17: note = ANti-CD19 CAR
AAAAAAAAAAAAAAAAA

SEQ ID NO: 8 - DNA, 23 bp, Homo sapiens
  source 1..23: mol_type = unassigned DNA
gagaatcaaaatcggtgaatagg

SEQ ID NO: 9 - DNA, 23 bp, Homo sapiens
  source 1..23: mol_type = unassigned DNA
ttcaaaacctgtcagtgattggg

SEQ ID NO: 10 - DNA, 23 bp, Homo sapiens
  source 1..23: mol_type = unassigned DNA
tgtgctagacatgaggtctatgg

SEQ ID NO: 11 - DNA, 23 bp, Homo sapiens
  source 1..23: mol_type = unassigned DNA
cgtcatgagcagattaaacccgg

SEQ ID NO: 12 - DNA, 23 bp, Homo sapiens
  source 1..23: mol_type = unassigned DNA
tcagggttctggatatctgtggg

SEQ ID NO: 13 - DNA, 23 bp, Homo sapiens
  source 1..23: mol_type = unassigned DNA
gtcagggttctggatatctgtgg

SEQ ID NO: 14 - DNA, 23 bp, Homo sapiens
  source 1..23: mol_type = unassigned DNA
ttcggaacccaatcactgacagg

SEQ ID NO: 15 - DNA, 23 bp, Homo sapiens
  source 1..23: mol_type = unassigned DNA
taaacccggccactttcaggagg

SEQ ID NO: 16 - DNA, 23 bp, Homo sapiens
  source 1..23: mol_type = unassigned DNA
aaagtcagatttgttgctccagg

SEQ ID NO: 17 - DNA, 23 bp, Homo sapiens
  source 1..23: mol_type = unassigned DNA
aacaaatgtgtcacaaagtaagg

SEQ ID NO: 18 - DNA, 23 bp, Homo sapiens
  source 1..23: mol_type = unassigned DNA
tggatttagagtctctcagctgg

SEQ ID NO: 19 - DNA, 23 bp, Homo sapiens
  source 1..23: mol_type = unassigned DNA
taggcagacagacttgtcactgg

SEQ ID NO: 20 - DNA, 23 bp, Homo sapiens
  source 1..23: mol_type = unassigned DNA
agctggtacacggcagggtcagg

SEQ ID NO: 21 - DNA, 23 bp, Homo sapiens
  source 1..23: mol_type = unassigned DNA
gctggtacacggcagggtcaggg

SEQ ID NO: 22 - DNA, 23 bp, Homo sapiens
  source 1..23: mol_type = unassigned DNA
tctctcagctggtacacggcagg

SEQ ID NO: 23 - DNA, 23 bp, Homo sapiens
  source 1..23: mol_type = unassigned DNA
tttcaaaacctgtcagtgattgg

SEQ ID NO: 24 - DNA, 23 bp, Homo sapiens
  source 1..23: mol_type = unassigned DNA
gattaaacccggccactttcagg

SEQ ID NO: 25 - DNA, 23 bp, Homo sapiens
  source 1..23: mol_type = unassigned DNA
ctcgaccagcttgacatcacagg

SEQ ID NO: 26 - DNA, 23 bp, Homo sapiens
  source 1..23: mol_type = unassigned DNA
agagtctctcagctggtacacgg

SEQ ID NO: 27 - DNA, 23 bp, Homo sapiens
  source 1..23: mol_type = unassigned DNA
ctctcagctggtacacggcaggg

SEQ ID NO: 28 - DNA, 23 bp, Homo sapiens
  source 1..23: mol_type = unassigned DNA
aagttcctgtgatgtcaagctgg

SEQ ID NO: 29 - DNA, 23 bp, Homo sapiens
  source 1..23: mol_type = unassigned DNA
atcctcctcctgaaagtggccgg

SEQ ID NO: 30 - DNA, 23 bp, Homo sapiens
  source 1..23: mol_type = unassigned DNA
tgctcatgacgctgcggctgtgg

SEQ ID NO: 31 - DNA, 23 bp, Homo sapiens
  source 1..23: mol_type = unassigned DNA
acaaaactgtgctagacatgagg

SEQ ID NO: 32 - DNA, 23 bp, Homo sapiens
  source 1..23: mol_type = unassigned DNA
atttgtttgagaatcaaaatcgg

SEQ ID NO: 33 - DNA, 23 bp, Homo sapiens
  source 1..23: mol_type = unassigned DNA
catcacaggaactttctaaaagg

SEQ ID NO: 34 - DNA, 23 bp, Homo sapiens
  source 1..23: mol_type = unassigned DNA
gtcgagaaaagctttgaaacagg

SEQ ID NO: 35 - DNA, 23 bp, Homo sapiens
  source 1..23: mol_type = unassigned DNA
ccactttcaggaggaggattcgg

SEQ ID NO: 36 - DNA, 23 bp, Homo sapiens
  source 1..23: mol_type = unassigned DNA
ctgacaggttttgaaagtttagg

SEQ ID NO: 37 - DNA, 23 bp, Homo sapiens
  source 1..23: mol_type = unassigned DNA
agctttgaaacaggtaagacagg

SEQ ID NO: 38 - DNA, 23 bp, Homo sapiens
  source 1..23: mol_type = unassigned DNA
tggaataatgctgttgttgaagg

SEQ ID NO: 39 - DNA, 23 bp, Homo sapiens
  source 1..23: mol_type = unassigned DNA
agagcaacagtgctgtggcctgg

SEQ ID NO: 40 - DNA, 23 bp, Homo sapiens
  source 1..23: mol_type = unassigned DNA
ctgtggtccagctgaggtgaggg

SEQ ID NO: 41 - DNA, 23 bp, Homo sapiens
  source 1..23: mol_type = unassigned DNA
ctgcggctgtggtccagctgagg

SEQ ID NO: 42 - DNA, 23 bp, Homo sapiens
  source 1..23: mol_type = unassigned DNA
tgtggtccagctgaggtgagggg

SEQ ID NO: 43 - DNA, 23 bp, Homo sapiens
  source 1..23: mol_type = unassigned DNA
cttcttccccagcccaggtaagg

SEQ ID NO: 44 - DNA, 23 bp, Homo sapiens
  source 1..23: mol_type = unassigned DNA
acacggcagggtcagggttctgg

SEQ ID NO: 45 - DNA, 23 bp, Homo sapiens
  source 1..23: mol_type = unassigned DNA
cttcaagagcaacagtgctgtgg

SEQ ID NO: 46 - DNA, 23 bp, Homo sapiens
  source 1..23: mol_type = unassigned DNA
ctggggaagaaggtgtcttctgg

SEQ ID NO: 47 - DNA, 23 bp, Homo sapiens
  source 1..23: mol_type = unassigned DNA
tcctcctcctgaaagtggccggg

SEQ ID NO: 48 - DNA, 23 bp, Homo sapiens
  source 1..23: mol_type = unassigned DNA
ttaatctgctcatgacgctgcgg

SEQ ID NO: 49 - DNA, 23 bp, Homo sapiens
  source 1..23: mol_type = unassigned DNA
acccggccactttcaggaggagg

SEQ ID NO: 50 - DNA, 23 bp, Homo sapiens
  source 1..23: mol_type = unassigned DNA
ttcttccccagcccaggtaaggg

SEQ ID NO: 51 - DNA, 23 bp, Homo sapiens
  source 1..23: mol_type = unassigned DNA
cttacctgggctggggaagaagg

SEQ ID NO: 52 - DNA, 23 bp, Homo sapiens
  source 1..23: mol_type = unassigned DNA
gacaccttcttccccagcccagg

SEQ ID NO: 53 - DNA, 23 bp, Homo sapiens
  source 1..23: mol_type = unassigned DNA
gctgtggtccagctgaggtgagg

SEQ ID NO: 54 - DNA, 23 bp, Homo sapiens
  source 1..23: mol_type = unassigned DNA
ccgaatcctcctcctgaaagtgg

SEQ ID NO: 55 - DNA, 49 bp, Homo sapiens
  source 1..49: mol_type = unassigned DNA
ttccctcccaggcagctcacagtgtgccaccatggagttggggccccta

SEQ ID NO: 56 - DNA, 49 bp, Homo sapiens
  source 1..49: mol_type = unassigned DNA
tgcctctaccacttctatgaccagatggacctggctggagaagaagaga

SEQ ID NO: 57 - DNA, 49 bp, Homo sapiens
  source 1..49: mol_type = unassigned DNA
tcttcatccaagggacttttcctcccagaacccgacacagacaccatca

SEQ ID NO: 58 - DNA, 49 bp, Homo sapiens
  source 1..49: mol_type = unassigned DNA
tgttgtgtgacatggaaggtgatgaagagaccagggaggcttatgccaa

SEQ ID NO: 59 - DNA, 47 bp, Homo sapiens
  source 1..47: mol_type = unassigned DNA
ccaaagattcaggtttactcacgtcatccagcagagaatggaaagtc

SEQ ID NO: 60 - DNA, 49 bp, Homo sapiens
  source 1..49: mol_type = unassigned DNA
ttgtcccacagatatccagaaccctgaccctgccgtgtaccagctgaga

SEQ ID NO: 61 - DNA, 49 bp, Homo sapiens
  source 1..49: mol_type = unassigned DNA
tgtgtttgagccatcagaagcagagatctcccacacccaaaaggccaca

SEQ ID NO: 62 - DNA, 50 bp, Homo sapiens
  source 1..50: mol_type = unassigned DNA
ttcccacccgaggtcgctgtgtttgagccatcagaagcagagatctccca

SEQ ID NO: 63 - DNA, 49 bp, Homo sapiens
  source 1..49: mol_type = unassigned DNA
tttagaaagttcctgtgatgtcaagctggtcgagaaaagctttgaaaca

SEQ ID NO: 64 - DNA, 49 bp, Homo sapiens
  source 1..49: mol_type = unassigned DNA
tccagtgacaagtctgtctgcctattcaccgattttgattctcaaacaa

SEQ ID NO: 65 - DNA, 49 bp, Homo sapiens
  source 1..49: mol_type = unassigned DNA
tatatcacagacaaaactgtgctagacatgaggtctatggacttcaaga

SEQ ID NO: 66 - DNA, 49 bp, Homo sapiens
  source 1..49: mol_type = unassigned DNA
tgaggtctatggacttcaagagcaacagtgctgtggcctggagcaacaa

SEQ ID NO: 67 - DNA, 2814 bp, synthetic construct
  misc_feature 1..2814: note = TALE-nuclease sequence
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 68 - DNA, 2832 bp, synthetic construct
  misc_feature 1..2832: note = TALE-nuclease sequence
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 69 - DNA, 2814 bp, synthetic construct
  misc_feature 1..2814: note = TALE-nuclease sequence
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 70 - DNA, 2832 bp, synthetic construct
  misc_feature 1..2832: note = TALE-nuclease sequence
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 71 - DNA, 2814 bp, synthetic construct
  misc_feature 1..2814: note = TALE-nuclease sequence
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 72 - DNA, 2832 bp, synthetic construct
  misc_feature 1..2832: note = TALE-nuclease sequence
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 73 - DNA, 2814 bp, synthetic construct
  misc_feature 1..2814: note = TALE-nuclease sequence
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 74 - DNA, 2832 bp, synthetic construct
  misc_feature 1..2832: note = TALE-nuclease sequence
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 75 - DNA, 21 bp, synthetic construct
  misc_feature 1..21: note = PCR primer
  source 1..21: mol_type = other DNA
atcactggcatctggactcca

SEQ ID NO: 76 - DNA, 22 bp, synthetic construct
  misc_feature 1..22: note = PCR primer
  source 1..22: mol_type = other DNA
agagcccctaccagaaccagac

SEQ ID NO: 77 - DNA, 22 bp, synthetic construct
  misc_feature 1..22: note = PCR primer
  source 1..22: mol_type = other DNA
ggacctagtaacataattgtgc

SEQ ID NO: 78 - DNA, 20 bp, synthetic construct
  misc_feature 1..20: note = PCR primer
  source 1..20: mol_type = other DNA
cctcatgtctagcacagttt

SEQ ID NO: 79 - DNA, 21 bp, synthetic construct
  misc_feature 1..21: note = PCR primer
  source 1..21: mol_type = other DNA
accagctcagctccacgtggt

SEQ ID NO: 80 - DNA, 58 bp, Homo sapiens
  source 1..58: mol_type = unassigned DNA
tctcgctccgtggccttagctgtgctcgcgctactctctctttctggcctggaggcta

SEQ ID NO: 81 - DNA, 2814 bp, synthetic construct
  misc_feature 1..2814: note = Beta2M T01- TALEN - LEFT
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 82 - DNA, 2832 bp, synthetic construct
  misc_feature 1..2832: note = Beta2M T01 TALEN -RIGHT
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 83 - DNA, 50 bp, synthetic construct
  misc_feature 1..50: note = B2M T02- TALEN targeting sequence
  source 1..50: mol_type = other DNA
tccaaagattcaggtttactcacgtcatccagcagagaatggaaagtcaa

SEQ ID NO: 84 - DNA, 2814 bp, synthetic construct; identical to SEQ ID NO: 67
  misc_feature 1..2814: note = Beta2M T02-TALEN - LEFT
  source 1..2814: mol_type = other DNA

SEQ ID NO: 85 - DNA, 2832 bp, synthetic construct; identical to SEQ ID NO: 68
  misc_feature 1..2832: note = Beta2M T02-TALEN RIGHT
  source 1..2832: mol_type = other DNA

SEQ ID NO: 86 - DNA, 47 bp, synthetic construct
  misc_feature 1..47: note = B2M T03- TALEN targeting sequence
  source 1..47: mol_type = other DNA
ttagctgtgctcgcgctactctctctttctggcctggaggctatcca

SEQ ID NO: 87 - DNA, 2814 bp, synthetic construct
  misc_feature 1..2814: note = Beta2M T03-TALEN - LEFT
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 88 - DNA, 2832 bp, synthetic construct
  misc_feature 1..2832: note = Beta2M T03-TALEN -RIGHT
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 89 - protein, 526 aa, synthetic construct [sequence omitted]

SEQ ID NO: 90 - protein, 310 aa, synthetic construct
  REGION 1..310: note = SP-MICAed
MGGVLLTQRTLLSLVLALLFPSMASMEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 91 - protein, 313 aa, synthetic construct
  REGION 1..313: note = SP-MICBed
MGGVLLTQRTLLSLVLALLFPSMASMAEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 92 - protein, 209 aa, synthetic construct
  REGION 1..209: note = SP-ULBP1ed
MGGVLLTQRTLLSLVLALLFPSMASMGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPT

SEQ ID NO: 93 - protein, 211 aa, synthetic construct
  REGION 1..211: note = SP-ULBP2ed
MGGVLLTQRTLLSLVLALLFPSMASMGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAG

SEQ ID NO: 94 - protein, 206 aa, synthetic construct
  REGION 1..206: note = SP-ULBP3ed
MGGVLLTQRTLLSLVLALLFPSMASMDAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPT

SEQ ID NO: 95 - protein, 221 aa, synthetic construct
  REGION 1..221: note = SP-N2DL4ed
MGGVLLTQRTLLSLVLALLFPSMASMHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPD

SEQ ID NO: 96 - protein, 224 aa, synthetic construct
  REGION 1..224: note = SP-RET1Ged
MGGVLLTQRTLLSLVLALLFPSMASMGLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPR

SEQ ID NO: 97 - protein, 211 aa, synthetic construct
  REGION 1..211: note = SP-RAETILed
MGGVLLTQRTLLSLVLALLFPSMASMRRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAG